(19) United States
(12) Patent Application Publication
(10) Pub. No.: US 2026/0102432 A1
(43) Pub. Date: Apr. 16, 2026

(54) COMPOSITION FOR CANCER IMMUNOTHERAPY COMPRISING SECONDARY LYMPHOID ORGAN-DERIVED EXTRACELLULAR VESICLE AS ACTIVE INGREDIENT

(71) Applicants: KOREA INSTITUTE OF SCIENCE AND TECHNOLOGY, Seoul (KR); Kyungpook National University Industry-Academic Cooperation Foundation, Daegu (KR)

(72) Inventors: In-San KIM, Seoul (KR); Jihoon HAN, Seoul (KR); Seong A. KIM, Seoul (KR); Eun Jung LEE, Daegu (KR); Wonkyung AHN, Daegu (KR); Na-yeon KIM, Seongnam-si (KR)

(73) Assignees: KOREA INSTITUTE OF SCIENCE AND TECHNOLOGY, Seoul (KR); Kyungpook National University Industry-Academic Cooperation Foundation, Daegu (KR)

(21) Appl. No.: 19/357,810

(22) Filed: Oct. 14, 2025

(30) Foreign Application Priority Data
Oct. 14, 2024 (KR) 10-2024-0139622

(51) Int. Cl. A61K 35/26; A61K 9/06; A61K 45/06; A61K 47/42; A61P 35/00

(52) U.S. Cl. CPC A61K 35/26; A61K 9/06; A61K 45/06; A61K 47/42; A61P 35/00

(57) ABSTRACT

The present disclosure relates to use of a secondary lymphoid organ (SLO)-derived extracellular vesicle (EV) for induction of immune cell activation, induction of ectopic lymphoid structure formation, cancer immunotherapy, and the like. The SLO-derived EV can be provided in a hydrogel formulation to induce formation of ectopic lymphoid structures. The SLO-derived EV may be used together with immune checkpoint inhibitors to exhibit higher anticancer activity by inducing immune cell activation. Furthermore, the SLO-derived EV may exhibit anticancer activity together with immune checkpoint inhibitors in immune checkpoint inhibitor-resistant cancers. Therefore, the SLO-derived EV is expected to be used as an effective anticancer agent or anticancer adjuvant for cancer patients with resistance or tolerance to immune checkpoint inhibitors.

### CROSS-REFERENCE TO RELATED APPLICATION

This application claims the benefit of Korean Patent Application No. 10-2024-0139622, filed on Oct. 14, 2024, in the Korean Intellectual Property Office, the entire disclosure of which is incorporated herein by reference for all purposes.

### BACKGROUND

### 1. Field of the Invention

One or more embodiments relate to use of a secondary lymphoid organ-derived extracellular vesicle for induction of immune cell activation, induction of ectopic lymphoid structure formation, cancer immunotherapy, and the like.

### 2. Description of the Related Art

Hydrogels are complex three-dimensional networks composed of physically or chemically crosslinked hydrophilic matrices, possessing high water content and tunable viscoelastic properties. These characteristics have attracted considerable attention in biomedical research. Among various hydrogel platforms composed of synthetic and natural polymers, protein-based hydrogels possess excellent biocompatibility and biodegradability. Such hydrogels can mimic natural tissues and incorporate various biological functions. Generally, the ‘liquid’ state of proteins is converted to a predominantly ‘solid’ hydrogel state through (i) physical crosslinking involving molecular self-assembly using short peptides such as β-hairpin peptides and α-helical coils and (ii) chemical crosslinking of elastic proteins of predominantly disordered proteins. However, in nature, numerous structural materials including wood, muscle, tendon, bone, and shells exhibit hierarchical order with delicate combinations and balance between structural and non-structural elements. The hierarchical structure of proteins achieves a balance between strength and robustness, playing a crucial role in adapting to complex environments including cell proliferation, differentiation, and motility. For example, the stability and elasticity of muscle are driven by complex molecular organization composed of intrinsically disordered sequences and series of individually folded immunoglobulin-like domains. Furthermore, the balance between folded and unfolded protein domains within the extracellular matrix (ECM) plays an important role in mediating various interactions between ECM-associated proteins and receptors and affecting mechanical properties.

However, most existing synthetic hydrogels exhibit predominantly isotropic structures because monomers of polymer chains with simple and identical chemical structures are uniformly distributed in aqueous solutions. Previous studies have proposed hybrid hydrogel materials incorporating protein nanocrystals into synthetic polymer networks (e.g., polyacrylate-acrylamide). Protein crystal structure hydrogels exhibited self-healing properties through excessive expansion and contraction of crystal lattices without losing their ability to be degraded or return to their original crystalline state. The aforementioned prior study emphasized the impact of protein crystal structures within hydrogels on functionality. However, the protein crystal structures used were not essential components of hydrogel formation but were simply incorporated into synthetic polymer hydrogels. Such synthetic polymer hydrogels are not suitable for in vivo applications and are insufficient to replicate nature's hierarchical structures. Recent efforts have focused on understanding the complex relationships between gel transitions occurring in vivo and the structure of individual component proteins, particularly chain entanglement.

Ectopic lymphoid structures (ELSs) are lymphocyte-like aggregates formed in non-lymphoid tissues during chronic inflammation such as infection, autoimmune diseases, and cancer, similar to tertiary lymphoid structures (TLSs). The ELSs obtained names similar to secondary lymphoid organs (SLOs) because they resemble immune cell aggregates and sometimes form germinal center-like structures. Since the presence of ELSs can be an indicator of good prognosis in various types of malignant tumors, increasing interest has promoted extensive research. Moreover, ELSs positively correlate with therapeutic responses to immunotherapy, particularly immune checkpoint blockade (ICB) therapy. Therefore, recent attempts have been made to artificially induce ELSs. However, most studies to date have focused only on the occurrence of ELSs, and research has been conducted by simply applying combinations of ELS-related molecules (LIGHT(TNFSF14)) or various cytokines such as CXCL12, CXCL13, CCL19, CCL21, Lymphotoxin α1β2, and sRANKL. Therefore, comprehensive investigation is needed to elucidate the impact of ELS neogenesis on tumor microenvironment (TME) and the ability to overcome ICB-related limitations through ideal ELS formation.

Extracellular vesicles (EVs) encompass various lipid bilayer vesicles secreted by living cells, including exosomes and ectosomes. Genetic material or proteins delivered by EVs can be taken up by recipient cells to alter their function and physiology, making EVs important means of communication between cells, tissues, and even organs. Consequently, EVs have attracted considerable attention, particularly in diagnostics and stem cell therapy. Also, EVs are drawing attention in the medical field because EVs retain characteristics derived from their parent cells. Similarly, organ- or tissue-derived EVs can reflect their parent cells or tissues. Particularly, recent studies have reported that adipose tissue-derived EVs play important roles in metabolic disorders such as obesity, adipose inflammation, and diabetes.

### SUMMARY

Embodiments provide secondary lymphoid organ (SLO)-derived extracellular vesicle (EV) for use in immune cell activation and/or induction of ectopic lymphoid structure formation.

Embodiments further provide a pharmaceutical composition for cancer immunotherapy including the SLO-derived EV, and provide the composition for use in combination with an immune checkpoint inhibitor.

However, technical goals to be achieved are not limited to those described above, and other goals not mentioned above are clearly understood by one of ordinary skill in the art from the following description.

According to an aspect, there is provided a composition for inducing ectopic lymphoid structure formation including SLO-derived EV as an active ingredient.

Furthermore, there is provided a composition for immune cell activation, including SLO-derived EV as an active ingredient.

Furthermore, there is provided a pharmaceutical composition for cancer immunotherapy, including SLO-derived EV as an active ingredient.

Furthermore, there is provided an anticancer composition for combination administration with an immune checkpoint inhibitor including SLO-derived EV as an active ingredient.

In an embodiment of the present disclosure, the SLO may be spleen, and the EV may be derived from spleen cells.

In another embodiment of the present disclosure, the SLO-derived EV may be provided in a state encapsulated in a hydrogel.

In another embodiment of the present disclosure, the hydrogel may be formed by crosslinking of ferritin-resilin (FR) proteins, and the ferritin protein may be human ferritin heavy chain protein.

In another embodiment of the present disclosure, the FR protein may be composed of ferritin protein having recombinant rec1-resilin linked to C-terminus of the ferritin protein, and the ferritin protein and rec1-resilin may be linked via a linker.

In another embodiment of the present disclosure, the linker may include or consist of an amino acid sequence of SEQ ID NO: 1, and may include or consist of an amino acid sequence in which an amino acid sequence of SEQ ID NO: 1 is repeated 1 to 5 times. More specifically, the linker may include or consist of an amino acid sequence in which an amino acid sequence of SEQ ID NO: 1 is repeated 2 to 4 times or 3 times.

In another embodiment of the present disclosure, the FR protein may have a CP05 peptide fused to C-terminus of the FR protein, and the CP05 peptide may include or consist of an amino acid sequence of SEQ ID NO: 2.

In another embodiment of the present disclosure, the FR protein may include [human ferritin heavy chain—rec1-resilin—CP05] in order from N-terminus to C-terminus.

In another embodiment of the present disclosure, the ferritin region in the FR protein may include mutations in one or more amino acids within a range that maintains self-assembly activity. The mutations may be interpreted to include deletions, substitutions, and insertions.

In another embodiment of the present disclosure, the hydrogel may be formed by heat treating a solution containing FR protein at a concentration exceeding 5 weight percent (wt %) at a temperature of 50° C. or higher. The solvent of the solution containing FR protein may be distilled water or phosphate-buffered saline (PBS). More specifically, the hydrogel may be formed by heat treating a 10-20 wt % FR solution at a temperature of about 55° C. for 3 minutes or longer.

In another embodiment of the present disclosure, the SLO-derived EV may be provided in a form encapsulated in nanocages formed by self-assembly ability and crosslinking of the aforementioned FR proteins.

Furthermore, there is provided a method of preparing an FR nanostructure including SLO-derived EV. The method includes the following steps of (1) preparing an FR protein having a structure of [N-terminus—human ferritin heavy chain—linker—Rec1-resilin—CP05 peptide—C-terminus], (2) mixing the FR protein with distilled water or PBS to prepare an FR solution, and (3) mixing EV isolated from SLO with a 10-20 wt % FR solution.

In one embodiment of the present disclosure, the preparation method may further include (4) performing heat treatment on a mixed solution of step (3) to provide an FR nanostructure in a hydrogel formulation.

In another embodiment of the present disclosure, the heat treatment of step (4) may include applying heat of 50° C. or higher for 3 minutes or longer.

Furthermore, there is provided an FR nanostructure including SLO-derived EV prepared by the aforementioned method.

Furthermore, there is provided use of the FR nanostructure for immune cell activation, Ectopic lymphoid structure (ELS) formation induction, and/or anticancer applications.

Furthermore, there is provided use for combination administration of an anticancer composition including the FR nanostructure as an active ingredient with an immune checkpoint inhibitor.

Additional aspects of embodiments will be set forth in part in the description which follows and, in part, will be apparent from the description, or may be learned by practice of the disclosure.

According to embodiments, the present disclosure may induce formation of ELSs by SLO-derived EV. The SLO-derived EV may be used together with immune checkpoint inhibitors to exhibit higher anticancer activity by inducing immune cell activation. Furthermore, the SLO-derived EV may exhibit anticancer activity together with immune checkpoint inhibitors in immune checkpoint inhibitor-resistant cancers. Therefore, the SLO-derived EV is expected to be used as an effective anticancer agent or anticancer adjuvant for cancer patients with resistance to immune checkpoint inhibitors.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIGS. 1A and 1B are schematic diagrams showing that designed hierarchical protein hydrogel loads spleen extracellular vesicles (sEVs) to induce ectopic lymphoid structure (ELS) formation, thereby recruiting immune cells to suppress tumor growth in tumor microenvironment (TME).

FIG. 1A is a schematic diagram illustrating the synthesis of EV-loaded hierarchical protein hydrogel. Specifically, monomer proteins composed of ferritin nanocages and intrinsically disordered rec1-resilin protein are functionalized with CP05 peptide capable of binding to sEVs, thereby self-assembling into spherical building block proteins and then forming hierarchical protein hydrogel through heat treatment.

FIG. 1B shows that in vivo injection of sEV-loaded hydrogel into tumor-bearing mice induces ELS formation abundantly containing T cells and B cells. ELS-inducing hydrogel promotes efficient immune response capable of overcoming cancer in combination with immune checkpoint blockade (ICB) therapy.

FIGS. 2A to 2H show results of characterization of biosynthesized ferritin-resilin (FR) building block protein plasmid expression vector and generated hydrogel that produce solid hierarchical hydrogel formulations from building block solutions at concentrations of 20 weight percent (wt %), 10 wt %, and 5 wt %.

FIG. 2A is a schematic diagram illustrating plasmid expression vector of FR building block protein in recombinant Escherichia coli (E. coli).

FIGS. 2B to 2D show analysis of expression levels of soluble(S) and insoluble (IS) fractions of FR building block protein synthesized in E. coli cytoplasm through (FIG. 2B) sodium dodecyl sulfate-polyacrylamide gel electrophoresis (SDS-PAGE) analysis, (FIG. 2C) Western blot analysis, and (FIG. 2D) purification using Ni+2 affinity column chromatography. Before SDS-PAGE and Western blot analysis, cell lysates were centrifuged at 13,000 rpm to separate S and IS fractions (M: molecular marker, BL21: wild-type E. coli BL21 (DE3) cell lysate).

FIG. 2E shows size exclusion chromatography (SEC) elution profile of purified FR building block protein indicating homogeneous nano-sized multimeric particles.

FIG. 2F shows representative photographs of 20 wt % (200 mg/mL), 10 wt % (100 mg/mL), and 5 wt % (50 mg/mL) FR building block protein solutions before and after heat treatment. (DW: distilled water used as control)

FIGS. 2G and 2H show that hierarchical protein hydrogels containing 20 wt %, 10 wt %, and 5 wt % building block protein concentrations were crosslinked through di- or tri-tyrosine bonds, exhibiting blue autofluorescence under UV illumination (FIG. 2G), with quantification of relative fluorescence units shown in (FIG. 2H) (***p<0.001, compared to DW control) (n=3 per group).

FIGS. 3A to 3G show results of physicochemical properties of FR building block protein and hydrogelation by heat treatment.

FIGS. 3A and 3B show transmission electron microscopy (TEM) images and dynamic light scattering (DLS) analysis indicating homogeneous spherical nano-sized cage structures of (FIG. 3A) purified wild-type ferritin (wtFTN) and (FIG. 3B) FR building block protein, respectively, showing different diameters (scale bar: 50 nm) (n=3 per group).

FIG. 3C shows representative photographs showing heat-induced changes of wtFTN, rec 1-resilin, and FR protein solutions before and after heat treatment. Each panel is provided with schematic diagrams of intramolecular and intermolecular crosslinked polymer networks.

FIG. 3D shows a representative photograph of FR hydrogel with wtFTN, rec1-resilin, and DW under UV illumination, showing blue autofluorescence due to di- or tri-tyrosine crosslinking. (Graph: fluorescence quantification of each sample) (n=3 per group)

FIG. 3E shows a representative photograph and a schematic diagram of individually synthesized human ferritin nanocage and rec1-resilin protein after heat treatment, showing that solid hydrogel formation does not occur.

FIGS. 3F and 3G show representative photographs and schematic diagrams of mutant hierarchical FR protein hydrogels based on (FIG. 3F) cysteine-removed C90R and (FIG. 3G) non-peroxidase reactive C90R/C102A/C130A building block proteins.

FIGS. 4A to 4C show formation of 10 wt % and 20 wt % hierarchical FR hydrogels heat-treated at 37° C.

FIG. 4A shows a photograph of 20 wt % FR building block protein solution after heat treatment at 37° C. (Left: distilled water used as control)

FIGS. 4B and 4C show self-healing tests of (FIG. 4B) 10 wt % and (FIG. 4C) 20 wt % hierarchical protein hydrogels, demonstrating (i) FR solution before heat treatment, (ii) FR solution after heat treatment, (iii) fragmentation of solid hydrogel, and (iv) hydrogel reformation after additional heat treatment.

FIGS. 5A to 5K show results of characterization of hierarchical FR protein hydrogel.

FIGS. 5A and 5B show representative oscillatory rheological characterization results of FR hydrogel: (FIG. 5A) temperature sweep and (FIG. 5B) time sweep at 55° C. (Solid line: storage modulus G′, Dashed line: loss modulus G″)

FIGS. 5C and 5D show representative thermal transition results of FR building block protein: (FIG. 5C) differential scanning calorimetry (DSC) and (FIG. 5D) Fourier transform infrared spectroscopy (FT-IR) analysis. (Pristine: FR solution before heat treatment)

FIGS. 5E and 5F show representative scanning electron microscopy (SEM) images showing microstructures of 5 wt %, 10 wt %, and 20 wt % FR hydrogels: (FIG. 5E) interconnected porous three-dimensional polymer network and (FIG. 5F) linear arrangement of 10 wt % hierarchical FR hydrogel.

FIG. 5G shows representative AFM surface morphology images of 1 wt % FR solution before and after heat treatment. (Graph: lateral size quantification of particles) (n=3)

FIGS. 5H and 5I show results of swelling kinetics analysis of 10 wt % and 20 wt % FR hydrogels: (FIG. 5H) swelling ratio and (FIG. 5I) water content measurement at day 1 and day (equilibrium state). (n=3 per group)

FIGS. 5J and 5K show self-healing test: experiments for (FIG. 5J) 10 wt % and (FIG. 5K) 20 wt % FR hydrogels, demonstrating (i) FR solution, (ii) heat-treated FR hydrogel, (iii) fragmentation of solid hydrogel, and (iv) reformation of hydrogel after additional heat treatment. (n=3 per group)

FIGS. 6A and 6B show results of sEV protocol selection and cytokine array analysis. FIG. 6A shows Western blot analysis for major EV markers (TSG101, CD63, CD81, and Alix) and negative marker (calnexin) of sEVs obtained through three different types of protocols (P1, P2, and P3).

FIG. 6B shows representative images of Proteome Profiler Mouse XL Cytokine array membrane of muscle extracellular vesicle (mEV) and sEV.

FIGS. 7A to 7J show isolation and characterization results of binding, loading, and release capabilities of sEVs to FR-CP05 hydrogel.

FIG. 7A shows Western blot analysis of cell lysate (CL) and EVs detecting EV markers (Alix, TSG101, and CD81) and negative marker (Calnexin) for samples derived from spleen and skeletal muscle.

FIGS. 7B and 7C show nanoparticle tracking analysis (NTA) and cryo-TEM images showing size distribution, particle count, and spherical nano-sized particle structure of isolated sEV. (Scale bar: 200 nm) (n=4)

FIG. 7D shows analysis results of EV-loaded proteins using Proteome Profiler Mouse XL Cytokine Array, showing heatmap profiles of mEV and sEV based on relative abundance for three categories of proteins directly related to ELS induction, particularly cytokines. (n=3 per group)

FIG. 7E shows TEM image (left) and DLS analysis (right) of purified FR-CP05 building block protein showing homogeneous spherical nano-sized cage structure. (Scale bar: 50 nm) (n=3)

FIG. 7F shows representative photographs (left) and an SEM image (right) showing changes of FR-CP05 protein solution before and after heat treatment.

FIGS. 7G and 7H show schematic diagram and flow cytometry analysis of binding experiments of FR-CP05 and wtFTN to sEVs. sEV-coated beads were incubated with wtFTN or FR-CP05 (10 mg/ml), and then ferritin-positive beads were analyzed by flow cytometry (left). Various doses of FR-CP05 show significant dose-dependent binding profile compared to wtFTN for sEV-coated beads (right). (n=4 per group)

FIG. 7I shows analysis of loading level of Cy5-labeled sEV within FR-CP05 hydrogel. (n=3)

FIG. 7J shows time-dependent release analysis of Cy5-labeled sEV loaded within FR or FR-CP05 hydrogel. (n=3 per group)

FIGS. 8A to 8I show results of fabrication and characterization of biosynthesized FR-CP05 building block protein and its solid hierarchical FR-CP05 hydrogel.

FIG. 8A is a schematic diagram illustrating plasmid expression vector of FR building block protein in recombinant E. coli.

FIGS. 8B to 8D show analysis of expression levels of S and IS fractions of FR-CP05 building block protein synthesized in E. coli cytoplasm through (FIG. 8B) SDS-PAGE analysis and (FIG. 8C) Western blot analysis, and (FIG. 8D) purification using Ni+2 affinity column chromatography. Before SDS-PAGE and Western blot analysis, CLs were centrifuged at 13,000 rpm to separate S and IS fractions (M: molecular marker, BL21: wild-type E. coli BL21 (DE3) CL).

FIGS. 8E and 8F show that blue autofluorescence due to di- or tri-tyrosine bonds of FR-CP05 protein hydrogel was observed under (FIG. 8E) UV illumination, and (FIG. 8F) relative fluorescence units were quantified compared to DW, wtFTN, and rec1-resilin (n=3 per group).

FIGS. 8G and 8H show results of swelling kinetics analysis of FR-CP05 hydrogel: (FIG. 8G) swelling ratio and (FIG. 8H) water content measurement results at day 1 and day 10 (equilibrium state) (n=3 per group).

FIG. 8I shows self-healing test of FR-CP05 hydrogel showing (i) solid hydrogel formation at 55° C., (ii) after fragmentation, and (iii) reformation after additional heat treatment.

FIG. 9 shows flow cytometry analysis results for aldehyde/sulfate latex beads.

Aldehyde/sulfate latex beads were coated with sEVs and then treated with wtFTN or FTN-CP05. Subsequently, the treated beads were stained with CD63 or ferritin antibody. The beads were first gated using FSC-A and SSC-A profiles. Comparison of CD63-positive samples and negative samples showed clear differences between latex beads without sEVs (thick line) and latex beads with sEVs (thin line), confirming that the beads with sEVs were distinctly coated with CD63-positive sEVs. Additional gating of ferritin-positive beads revealed clear differences between wtFTN and FTN-CP05, and these differences were confirmed based on negative samples not coated with sEVs.

FIGS. 10A to 10H show evaluation of artificial ELS formation within sEV-loaded FR-CP05 hydrogel and immune cell profiling.

FIG. 10A is a schematic diagram of the schedule for implanting hydrogel in mice. Hydrogel was removed and stained several days after injection.

FIGS. 10B and 10C show representative photographs of (FIG. 10B) hydrogel formation in vivo and (FIG. 10C) extracted hydrogel 10 days after injection. Black arrows indicate angiogenesis in sEV-loaded hydrogel.

FIG. 10D shows representative images of hematoxylin and eosin (H&E) staining of extracted hydrogel (Upper photographs are 10× magnification and lower photographs are 40× magnification).

FIGS. 10E to 10H show analysis of artificial ELS formation by sEV-loaded hydrogel using multiplex immunohistochemistry (IHC). (FIG. 10E) Representative multiplex images of sEV-loaded hydrogel stained with B220 (dark gray), CD8 (medium gray), CD4 (light gray), CD11c (very dark gray), CD11b (gray darker than medium gray), and CD31 (light-to-medium gray) (scale bar: 200 μm). (FIG. 10F) Quantification of cells infiltrated within sEV-loaded hydrogel. (FIG. 10G) Representative images of germinal center-like immune cell aggregates composed of B220+ cells, CD4+ cells, and CD8+ cells (scale bar: 50 μm). (FIG. 10H) Median nearest neighbor distance from B220+ cells to each cell class (n=2).

FIGS. 11A and 11B show results confirming artificial ELS formation (angiogenesis and recruitment of various cells).

FIG. 11A shows that hydrogel loaded with sEVs demonstrates angiogenesis potential, assessed by the presence of red blood cells indicated by black arrows.

FIG. 11B shows quantification results of stained cells detected in hydrogel.

FIGS. 12A to 12F show results confirming antitumor effects of artificial ELS formation induced by sEVs-loaded hydrogel together with ICB therapy.

FIG. 12A shows mean tumor growth curves and individual tumor growth curves of each group. (n=11-14 per group)

FIGS. 12B to 12E show results confirming the number of cells infiltrated within tumor tissue (per mm³).

FIG. 12B Representative flow cytometry plots showing number of CD4+ T cells (CD45.2+CD3+CD4+) and CD8+ T cells (CD45.2+CD3+CD8+), and CD8+ T cell subsets containing Granzyme B+ cells in each group. (n=6-9 per group)

FIG. 12C Number of Granzyme B+CD8+ T cells (CD45.2+CD3+CD8+GranzymeB+) in tumors of each group.

FIG. 12D Number of Treg cells (CD45.2+CD3+CD4+FoxP3+) in tumors of each group. (n=4-6 per group)

FIG. 12E Number of B220+ B cells (CD45.2+B220+) in tumors of each group and representative flow cytometry plots of CD45R/B220+ B cells within CD45.2+ cell subsets. (n=8-12 per group)

FIG. 12F shows representative photographs of multiplex images stained with CD20 (dark gray), CD8 (medium gray), and CD4 (light gray) in tumors.

FIG. 13 shows flow cytometry analysis results for tumor cell analysis.

Tumor cells were isolated into single cells, and dead cells were removed. The cells were then fixed and permeabilized, stained with antibodies, and examined by flow cytometry. The cells were first gated using FSC-A and SSC-A profiles. Doublet cells were removed using FSC-A and FSC-H. Immune cells were selected by gating CD45.2+ cells and then further analyzed for CD3+, CD4+, and CD8+ T cells, as well as FoxP or Granzyme B. B220 was used to identify B220+ B cells.

### DETAILED DESCRIPTION

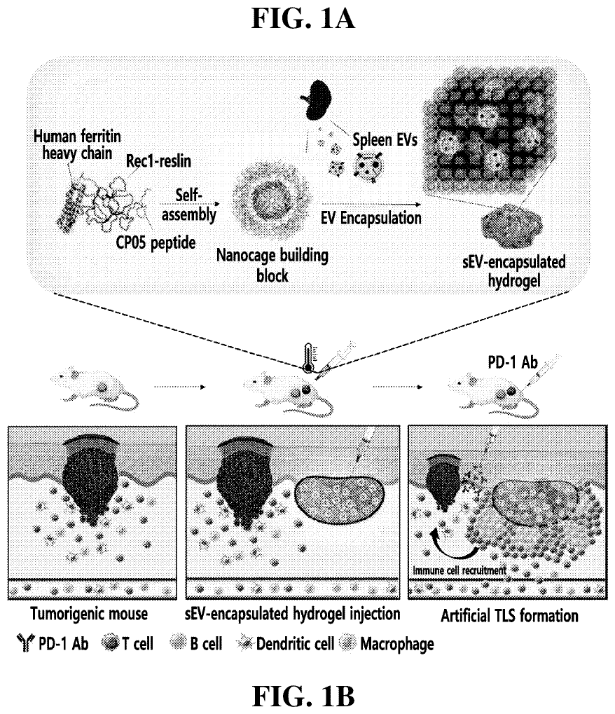

In this study, a novel type of hydrogel that has a hierarchical superstructure capable of loading secondary lymphoid organ (SLO)-derived extracellular vesicle (EV) to promote ectopic lymphoid structure (ELS) formation was developed (FIG. 1). The hydrogel combines self-assembling ferritin nanocages that form highly organized supramolecular crystal structures with disordered and flexible elastic rec1-resilin protein to form hierarchical nanocomposite structures. The protein hydrogel possesses biocompatibility, biodegradability, sufficient mechanical stability, and high water content. Additionally, the protein hydrogel has self-healing properties, making the protein hydrogel suitable as an injectable hydrogel. The hydrogel may load SLO-derived EV by incorporating EV-binding peptides, thereby inducing ELS formation within or near the tumor microenvironment (TME). Therefore, the combination of ELS formation induction and immune checkpoint blockade (ICB) therapy enhances immune responses to overcome tumor burden.

Accordingly, the present disclosure provides SLO-derived EV for use in immune cell activation and/or induction of ELS formation.

SLOs include lymph nodes, spleen, Peyer's patches (PPs), and mucosal tissues including nasal associated lymphoid tissue (NALT), adenoid, and tonsil. In specific embodiments of the present disclosure, spleen-derived EV was used.

Furthermore, the present disclosure provides a pharmaceutical composition for cancer immunotherapy including the SLO-derived EV, and the pharmaceutical composition is provided for use in combination with an immune checkpoint inhibitor.

On the other hand, the present disclosure may provide the SLO-derived EV in a form encapsulated in a hydrogel. The hydrogel may be formed by crosslinking of ferritin proteins, and the ferritin protein may be human ferritin heavy chain protein. A CP05 peptide sequence may be fused to the C-terminus of the ferritin protein.

The pharmaceutical composition for cancer immunotherapy of the present disclosure may be co-formulated for combination administration with an immune checkpoint inhibitor as a pharmaceutical formulation, or may be administered alternately and separately as a therapeutic combination. In this case, when administered alternately and separately, the pharmaceutical composition and the immune checkpoint inhibitor may be administered simultaneously or sequentially.

In the present disclosure, “simultaneously” means that the administration of the immune checkpoint inhibitor and the pharmaceutical composition of the present disclosure is performed within 24 hours, and “sequentially” means that the administration is performed over 24 hours.

The term “prevention” as used herein means any act of suppressing cancer or delaying the onset of cancer by administration of the pharmaceutical composition according to the present disclosure.

The term “treatment” as used herein means any act of improving cancer or beneficially altering the symptoms of cancer by administration of the pharmaceutical composition according to the present disclosure.

The pharmaceutical composition according to the present disclosure may further include pharmaceutically acceptable carriers, excipients, or diluents. Examples of pharmaceutically acceptable carriers, excipients, and diluents that can be used in the pharmaceutical composition of the present disclosure include lactose, dextrose, sucrose, sorbitol, mannitol, xylitol, erythritol, maltitol, starch, acacia gum, alginate, gelatin, calcium phosphate, calcium silicate, calcium carbonate, cellulose, methyl cellulose, polyvinylpyrrolidone, water, methylhydroxybenzoate, propylhydroxybenzoate, talc, magnesium stearate, mineral oil, and the like.

The pharmaceutical composition of the present disclosure may be administered orally or parenterally according to the desired method, but parenteral administration is preferable.

According to an embodiment of the present disclosure, the pharmaceutical composition according to the present disclosure may be administered directly intravenously, intraarterially, intratumorally, or subcutaneously, and may be administered as an injection. The injection according to the present disclosure may be in a form dispersed in a sterile medium so that the injection can be used as is when administered to a patient, or may be in a form administered after adding distilled water for injection to disperse the injection to an appropriate concentration upon administration. Furthermore, when prepared as an injection, the injection may be mixed with buffering agents, preservatives, analgesics, solubilizers, isotonic agents, stabilizers, and the like, and may be prepared in unit dose ampoule or multiple dose forms.

The dosage of the pharmaceutical composition of the present disclosure varies depending on the patient's condition and weight, degree of disease, drug form, route of administration, and time, but can be appropriately selected by those skilled in the art. In addition, the pharmaceutical composition according to the present disclosure may be used alone or in combination with adjuvant therapy methods such as surgical therapy.

The present disclosure can be modified in various ways and has various embodiments. Specific embodiments are illustrated in the drawings and described in detail in the detailed description below. However, this is not intended to limit the present disclosure to specific embodiments, but should be understood to include all changes, equivalents, or substitutes included in the idea and technical scope of the present disclosure. In the description of the present disclosure, detailed description of well-known related technology will be omitted when it is deemed that such description will cause ambiguous interpretation of the present disclosure.

### Experimental Method

### 1. Biosynthesis of Ferritin Nanocage Crystals Displaying Rec1-Resilin

Recombinant rec1-resilin and human ferritin heavy chain (hFTN-H) genes were synthesized through CosmoGenetech Co. Korea. The following gene clones were synthesized through extension polymerase chain reaction (PCR) using appropriate primers: 6×histidine::hFTN-H::Linker::Rec1-resilin, 6×histidine::hFTN-H, 6×histidine::Rec1-resilin, and 6×histidine::hFTN-H::Linker::Rec1-resilin::CP05. Here, Linker and CP05 represent (GGGGS) 3 and CRHSQMTVTSRL sequences, respectively. The corresponding gene clones were ligated into pT7-7 plasmid vector to generate pT7-ferritin-resilin (FR), pT7-hFTN, pT7-rec1-resilin, and pT7-FR-CP05 expression vectors. The modified vectors were introduced into Escherichia coli (E. coli) (BL21 (DE3)) to select for ampicillin resistance to perform FR, hFTN-H, rec1-resilin, and FR-CP05 recombinant protein expression. Additional mutation vectors were constructed using a gene mutation kit (Intron Korea), encoding 6×histidine::hFTN-H (C90R)::Linker::Rec1-resilin and 6×histidine::hFTN-H (C90R/C102A/C130A)::Linker::Rec1-resilin genes. Here, cysteines at amino acid positions 90, 102, and 130 of the hFTN-H gene were mutated to arginine, alanine, and alanine, respectively.

### 2. Recombinant Protein Expression and Purification

Recombinant E. coli cells were cultured in a Luria-Bertani (LB) liquid medium containing ampicillin (100 mg/L) at 37° C., and OD600 reached approximately 1.0. To this, 0.5 mM isopropyl β-D-1-thiogalactopyranoside (IPTG) was added to induce protein expression at 37° C. for 4 hours. After centrifugation, the cells were harvested, and the cell pellet was suspended in lysis buffer (1M Tris/HCl, 10 mM imidazole, 1 mM benzamidine, 0.1% Triton-X, 5 mM 2-mercaptoethanol, pH 7.4). The cells were then disrupted using a sonicator (Sonic and Materials INC, Newtown, Connecticut, USA). The recombinant protein was separated from the insoluble fraction containing cell debris and protein aggregates by centrifugation at 13,000 rpm for 20 minutes to obtain a cell debris-free supernatant. The recombinant protein contained in the cell debris-free supernatant was purified through Ni²⁺-affinity chromatography (Ni-NTA agarose and column, Qiagen, Hilden, Germany).

The purification procedure was as follows: (1) the column resin was washed with lysis buffer before sample loading, (2) after sample loading, binding was performed at 4° C. for 1 hour, followed by washing with 4 ml of wash buffer (1M Tris/HCl, 80 mM imidazole, 100 mM NaH₂PO₄, 1 mM benzamidine, 500 mM sodium chloride, 10 mM 2-mercaptoethanol, pH 7.4), and (3) the recombinant protein was eluted with 1.5 ml of elution buffer (1M Tris/HCl, 250 mM imidazole, 1 mM benzamidine, 0.1% Triton-X, 5 mM 2-mercaptoethanol, pH 7.4) and the buffer was exchanged with distilled water (DW).

The expression level (percentage of total cellular protein) and cytoplasmic solubility (ratio of soluble fraction to insoluble fraction) of the synthesized recombinant protein were analyzed by sodium dodecyl sulfate-polyacrylamide gel electrophoresis (SDS-PAGE) using 12% Tris-glycine precast gel (Invitrogen, Carlsbad, CA, USA) and Western blotting (Bio-Rad, Hercules, CA, USA) using anti-ferritin heavy chain primary antibody (ab65080, Abcam, UK) and anti-rabbit secondary antibody (ab97080, Abcam, UK).

### 3. Physicochemical Characterization of Building Block Proteins

Purified building block protein samples were negatively stained with 2% (w/v) ammonium molybdate solution or uranyl acetate solution, placed on carbon film-coated 200 square mesh copper grids (Electron Microscopy Science, Pennsylvania, USA), and dried in air for 1 hour. The morphology of wild-type ferritin (wtFTN), FR, and FR-CP05 building block proteins was analyzed using a bio transmission electron microscope (bio-TEM, Hitachi, Tokyo, Japan) operated at 100 kV. The multimeric forms of FR and FR-CP05 building block proteins were analyzed through a Superdex column (Superdex 200 10/300 GL, Cytiva, Marlborough, Massachusetts, USA) using size exclusion chromatography (SEC: Akta Purifier 100, Cytiva, Marlborough, Massachusetts, USA). The hydrodynamic size of the building block proteins was analyzed using dynamic light scattering (DLS) with a Zetasizer Nano ZS system (Malvern Instruments Ltd., Malvern, UK).

### 4. Formation of Hierarchical Protein Hydrogel

Freeze-dried recombinant protein powder samples were dissolved in DW or phosphate-buffered saline (PBS) according to each concentration (wt %). After complete dissolution, the samples were maintained at fixed temperatures between 37° C. and 55° C. for 30 minutes or 12 hours using a water bath (Polyscience, Philadelphia, USA) to form a final hydrogel. For confirmation of di- or tri-tyrosine crosslinking, each hydrogel sample was observed with an ultraviolet (UV) transilluminator (WUV-L50, Daihan Scientific, Korea) and measured using the built-in fluorometer of a DS-11 FX+ spectrophotometer/fluorometer (DeNovix, Wilmington, USA). Representative data represents at least three independent experiments (N≥3).

### 5. Oscillatory Rheology Analysis

Rheological characterization of hierarchical FR hydrogel was performed using an oscillatory rheometer equipped with a Peltier plate accessory (HR20, TA Instruments, Delaware, USA), applying parallel plate geometry with a diameter of 25 mm and a gap of 1 mm. To study thermal gelation behavior, temperature sweep tests of FR hydrogel were performed from 25° C. to 85° C. with a heating rate of 1° C./min, constant angular frequency of 5 rad/s, and 1% strain. Based on the gelation point data obtained from the temperature sweep test, thermal gelation kinetics were performed through time sweep tests at 55° C. for 50 minutes with a constant angular frequency of 10 rad/s and 1% strain.

6. Differential Scanning calorimetry (DSC)

Thermal properties of hierarchical FR hydrogel were determined using a DSC (Q20, TA Instruments, Delaware, USA) with a second heating cycle at a heating rate of 10° C./min and N2 flow rate of 50 ml/min. First, temperature and heat flow were calibrated with standard indium. Hydrogel samples were applied to a slide glass with a spatula and then sufficiently frozen at −75° C. Freeze-drying was performed using EYELA FDU-1100 (Tokyo Rikakikai Co. Ltd, Japan). The initial sample represented protein powder, and the final sample was heated from −60° C. to 250° C. at a rate of 10° C./min in equilibrium at 25° C. The total heat flow was then recorded.

### 7. Fourier Transform Infrared Spectroscopy (FT-IR)

Hydrogel samples were applied to a slide glass, heated to 100° C., and dried for 1 hour. The heated and dried hydrogel samples were ground and mixed with potassium bromide (KBr) powder to make analytical pellets. The KBr pellets were placed on the stage of a Fourier transform infrared spectrometer (FTIR, IR Prestige-21, Shimadzu, Japan) and examined in absorption mode over a wavelength range of 400 cm⁻¹ to 4000 cm⁻¹.

### 8. Atomic Force Microscopy (AFM) Analysis

AFM observation was performed using XE7 (Park Systems, Korea) in non-contact mode at room temperature in air with a PPP-NCHR (Park System, Korea) cantilever to observe morphological differences between solution and gel states of a 1 wt % FR protein solution. For sample preparation, dissolved FR protein solution samples before and after heat treatment were cast on silicon wafers and then spin-coated at 4000 rpm for 30 seconds to form a thin film for optimal imaging.

9. Scanning Electron Microscopy (SEM) analysis

Additionally, morphological characteristics of FR and FR-CP05 protein hydrogels were evaluated using a field emission scanning electron microscope (FE-SEM, Hitachi SU8220). Hydrogel samples were immersed in liquid nitrogen and then transferred to EYELA FDU-1100 (Tokyo Rikakikai Co. Ltd, Japan) for freeze-drying to enable mounting on SEM sample holders. The freeze-dried hydrogel samples were separated into multiple pieces horizontally and vertically, coated with platinum (Pt) at room temperature, and prepared for SEM visualization. All SEM observations were performed at an acceleration voltage of 5.0 kilovolts (kV), 10 milliamperes (mA).

### 10. Swelling Analysis

To measure the accurate dry weight of protein hydrogel, hydrogel samples were frozen using liquid nitrogen and freeze-dried using EYELA FDU-1100 (Tokyo Rikakikai Co. Ltd, Japan). After measuring the dry weight of the freeze-dried hydrogel, the samples were immersed in 5 mL of 1×PBS at 37° C. for day 1 and day 10. The swollen hydrogel was weighed with a microbalance after removing excess water with filter paper. The water content of these hydrogels was evaluated as water content ratio (WC) and swelling ratio at equilibrium (q). Here, Ws represents the weight of hierarchical protein hydrogel swollen in PBS, and Wd represents the weight of hierarchical protein hydrogel composite dried after swelling. Representative data represents at least three independent experiments (N≥3).

### 11. Self-Healing Analysis

Thermally induced hierarchical protein hydrogel formed at 55° C. (or 37° C.) for 30 minutes (or 12 hours) was physically broken using a syringe needle. The completely broken hydrogel was tested for reformation to a hydrogel state through additional heat treatment overnight at 55° C. or 37° C. using a water bath (Polyscience, Philadelphia, USA).

### 12. EV Preparation and Isolation

EVs were prepared using the following three protocols. The second protocol (P2) among the three protocols was determined to be most suitable and was used in the remaining studies.

### 12-1. Protocol 1 (P1)

Mouse spleens were removed, pulverized using a GentleMACS grinder (Miltenyi Biotec, Bergisch-Gladbach, Germany), and suspended in PBS. The cell suspension was subjected to a series of centrifugations (300×g, 10 minutes: 2000×g, 10 minutes; and 10,000×g, 30 minutes) to remove microvesicles and cell debris. The suspension was then filtered through a 0.22 μm filter (431118; Corning®), Corning, NY, USA) and centrifuged again at 150,000×g for 2 hours. The pellet was suspended in PBS containing protease inhibitor cocktail (PIC: 11697498001; Roche, Basel, Switzerland) and stored at 4° C.

### 12-2. Protocol 2 (P2)

Mouse spleen or skeletal muscle tissue was removed, cut into 1 mm³ pieces, and cultured in Roswell Park Memorial Institute medium (RPMI LM011-01: Welgene, Taipei, China) with exosome-depleted fetal bovine serum (FBS, exosome-depleted, A2720801, Gibco, Billings, Montana, USA) and 1% antibiotic-antimycotic solution (15240-062; Gibco) for 48 hours. The supernatant was collected and subjected to a series of centrifugations (300×g, 10 minutes: 2000×g, 10 minutes: 10,000×g, 30 minutes), then filtered through a 0.22 μm filter (431118; Corning®) and centrifuged again at 150,000×g for 2 hours. The pellet was reconstituted in PBS containing PIC (11697498001; Roche) and stored at 4° C.

### 12-3. Protocol 3 (P3)

Mouse spleen or skeletal muscle tissue was removed, cut into 1 mm³ pieces, and cultured in RPMI medium. Collagenase type II (17101015: ThermoFisher Scientific, Waltham, MA, USA) at 2 mg/mL and DNase (18047019, ThermoFisher Scientific) at 80 U/mL were added to the cells and incubated at 37° C. for 30 minutes. After a series of centrifugations (300×g, 10 min: 2000×g, 10 min: 10,000×g, 30 min), the supernatant was filtered through a 0.22 μm filter (431118: Corning®) and centrifuged again at 150,000×g for 2 hours. The pellet was reconstituted in PBS containing PIC (11697498001; Roche) and stored at 4° C.

### 13. Nanoparticle Tracking Analysis

The size and number of EVs were measured using Zeta View® (Particle Metrix, Meerbusch, Germany) and the corresponding software (Zeta View 8.02.28). After calibration with the Zeta View system using polystyrene beads (3090A, ThermoFisher Scientific), EVs were diluted, loaded for analysis, and analyzed to obtain size distribution and particle counts. The samples were analyzed at 11 different locations within the chamber.

### 14. Cryogenic Transmission Electron Microscopy (Cryo-TEM)

EVs were visualized using cryoelectron microscopy (Tecnai Systems, Philips, Holland). Purified EVs were placed on copper grids coated with a thin carbon film and then cryopreserved using Vitrobot (FP5350; FEI).

### 15. Immunoblotting Analysis

The protein concentration of the samples was quantified using a DC™ protein assay kit (5000111: Bio-Rad, Hercules, CA, USA). The samples were lysed using immunoprecipitation buffer (RIPA buffer, 9806S; Cell Signaling Technology, Danvers, MA, USA), and separated by SDS-PAGE. The samples were loaded onto sodium sulfate-polyacrylamide gels, separated by electrophoresis, and transferred to nitrocellulose membranes. After blocking with 5% skimmed milk, the membranes were incubated overnight at 4° C. with primary antibodies (Tsg 101 (sc-22774; Santa Cruz, Dallas, TX, USA), CD81 (sc-166029; Santa Cruz), CD63 (sc-5275; Santa Cruz), ALIX (sc-53540; Santa Cruz), or calnexin (ab22595; Abcam, Cambridge, United Kingdom)). The membranes were then washed five times for 5 minutes each in a solution containing Tris-buffered saline (TBS) and 0.05% Tween-20, and subsequently incubated for 1 hour at 20-25° C. with secondary antibodies: either anti-mouse peroxidase (1:3000; A4416; Sigma-Aldrich) or anti-rabbit peroxidase (1:3000; A0545; Sigma-Aldrich). The membranes were then cultured using ECL substrate (1705061; Bio-Rad) and visualized using the ChemiDoc™ Touch Imaging System (Bio-Rad).

### 16. Cytokine Array

To investigate cytokine expression in EVs, a total of 100 μg of EVs were lysed in RIPA buffer and analyzed using the Proteome Profiler Mouse Cytokine Array Panel A Kit (ARY006, R&D Systems, NE Minneapolis, MN, USA). Cytokine expression was determined by densitometry after subtracting the mean signal of the negative control using ImageJ software (National Institutes of Health, Laboratory for Optical and Computational Instrumentation). Values are expressed as relative abundance comparing skeletal muscle EVs to spleen EVs. Representative data represents at least three independent experiments (N≥3).

### 17. Ferritin Binding Assay

10 μg of splenic EVs were bound to 20 μl of sulfate latex beads (S37498; ThermoFisher Scientific) by incubation for 15 minutes at room temperature. After adding 1 mL of PBS, the EVs and beads were incubated overnight on a rotary test wheel at 4° C. After adding 110 μl of 1 M glycine, the mixture was incubated at room temperature for 30 minutes and centrifuged at 4000 rpm for 3 minutes. The supernatant was then discarded. The EV-coated beads were suspended in PBS/0.5% bovine serum albumin (BSA), mixed with different concentrations of FR or FR-CP05, and incubated at room temperature for 1 hour. The beads were washed three times and then incubated with CD63 antibody (1:100, sc-5275) or ferritin antibody (1:100, ab65080; Abcam). After washing the beads twice with PBS/0.5% BSA, secondary antibody (1:200; Alexa FluorR) 64AffiniPure Donkey Anti-Rabbit IgG (H+L): Jackson ImmunoResearch, West Grove, PA, USA) was added. After washing twice with PBS/0.5% BSA, the EV-coated beads stained with antibodies were analyzed by flow cytometry. Beads treated only with secondary antibody and not stained with primary antibody were read as controls. Representative data represents at least three independent experiments (N≥3).

### 18. Analysis of EV Loading Levels in Hydrogels

sEVs were labeled with Cyanine5 NHC ester (63020; Lumiprobe, Cockeysville, MD, USA). Free Cyanine5 was then removed using Zeba™ Spin Desalting Columns (A57762; ThermoFisher Scientific). FR-CP05 protein powder was dissolved in this Cyanine5-labeled sEV solution, and dispensed into 96 black wells (CLS3603; Corning®) at 200 μL each. The solution was heated at 55° C. for 30 minutes to gel. The prepared hydrogels were washed three times with deionized water and then analyzed using a SpectraMax i3x multi-mode microplate reader (Molecular devices, California, USA). Representative data represents at least three independent experiments (N≥3).

### 19. Hydrogel Release Analysis of EV

50 μL of sEV-loaded FR-CP05 hydrogel was prepared. 200 μL of PBS was added to the hydrogel samples in 96 black wells. sEV release was then measured over a period of time. The fluorescence of released sEVs was continuously counted using a GloMax® Discover microplate reader (Promega, Madison, Wisconsin, USA). Representative data represents at least three independent experiments (N≥3).

### 20. Hematoxylin and Eosin (H&E) Staining

Hydrogels were extracted from mice 10 days after implantation, fixed in formalin, and embedded in paraffin tissue blocks. The paraffin blocks were sectioned at 4 μm thickness. Slides were deparaffinized, rehydrated, stained with H&E, and then dehydrated. The stained slides were observed under a light microscope.

### 21. Animal Experiments

Six-week-old male BALB/c mice were purchased from Orient Bio and housed in a specific pathogen-free animal facility at the Korea Institute of Science and Technology (KIST: Seoul, Republic of Korea) under conditions of temperature 23±2° C., relative humidity 55±10%, and 12-hour light/dark cycle with free access to food and water. The animal experimental protocol was approved by the Institutional Animal Care and Use Committee of KIST (approval number: KIST-IACUC-2020-095).

### 22. Cell Culture

CT26 mouse colon cancer cell line was purchased from ATCC (Manassas, VA, USA) and cultured in RPMI 1640 (SH30255.01; Hyclone, Logan, UT, USA) medium containing 10% FBS (12483-020; Gibco, Billings, Montana, USA) and 1% antibiotic-antimycotic (15240-062; Gibco). The cells were cultured at 37° C. and 5% CO2.

### 23. Hydrogel Implantation and In Vivo Tumor Model

1×10⁶ CT26 cells were subcutaneously inoculated into the left flank of BALB/C mice. Five days after inoculation, the mice received implantation of PBS, hydrogel, sEV, or sEV-loaded hydrogel. The hydrogel was formed by adding 1 mg/mL sEV or PBS to FR-CP05 protein powder (200 mg/mL). The hydrogel was injected twice around the tumor with a volume of 50 μL each. Equal amounts of PBS, EV, or empty hydrogel were injected into respective groups. Anti-mouse PD-1 antibody (BP0146; Lebanon, NH, USA) was injected 5 times at 2-day intervals starting from 11 days after tumor inoculation. Tumor size and body weight of the mice were monitored for several days after inoculation. The mice were then euthanized after tumors were harvested. Tumor size was calculated using the formula (width)×(length)/2, and mice with tumor size exceeding 2,000 mm³ were euthanized.

### 24. Flow Cytometric Analysis

Tumors were extracted from experimental mice several days after tumor inoculation and enzymatically digested using a tumor dissociation kit (130-096-730; Miltenyi Biotec, Bergisch-Gladbach, Germany) and GentleMACS homogenizer (Miltenyi Biotec). The digested tumors were passed through a 40 μm strainer. Red blood cells were then removed using red blood cell lysis buffer (420301, BioLegend, San Diego, CA, USA). Dead cells were removed using a dead cell removal kit (130-090-101: Miltenyi Biotec), and the remaining living cells were suspended in RPMI containing serum. Living cells were fixed and permeabilized using CytoFix/CytoPerm kit (BD 554714; BD Biosciences, Franklin Lakes, NJ, USA) and then stained with fluorescent antibodies. The antibodies used were anti-CD45.2 (109830), anti-CD3 (100222), anti-CD8a (100706), anti-Granzyme B (372212), anti-CD4 (100510), anti-FoxP3 (320008), and anti-B220/CD45R (103251) purchased from BioLegend. Flow cytometric analysis was performed using a CytoFLEX flow cytometer (Beckman Coulter Life Sciences, Brea, CA, USA), and flow cytometric data were analyzed using FlowJo (v10) software (TreeStar, San Francisco, CA, USA) and CytExpert (v2.5) software (Beckman Coulter Life Sciences).

### 25. Multiplex Immunohistochemistry (IHC)

Hydrogels were extracted from mice on day 10 after implantation. Tumors were then extracted on day 21 after inoculation. All samples were fixed with formalin and embedded in paraffin tissue blocks. Paraffin blocks were sectioned to 4 μm thickness, and the prepared slides were stained, scanned, and analyzed at prismCDX Co. Ltd (Gyeonggi-do, Korea). The slides were heated in a dry oven at 60° C. for at least 1 hour. Multiplex immunofluorescence staining was then performed using Leica Bond Rx™ Automated Stainer (Leica Biosystems, Seoul, Korea). Briefly, the slides were deparaffinized with Leica Bond Dewax solution (#AR9222: Leica Biosystems), followed by antigen retrieval with Bond Epitope Retrieval (#AR9640; Leica Biosystems) for 30 minutes. Staining involved a blocking step using blocking solution (C0103; TheraNovis), followed by incubation with primary antibodies for 30 minutes and incubation with Goat Anti-Rabbit IgG HRP secondary antibody (ab214880; Abcam) for 10 minutes. The primary antibodies used were CD11c (97585: CST, Danvers, MA, USA), CD8a (98941; CST), CD31 (ab124432: Abcam), CD11b (ab133357; Abcam), CD20 (ab64088; Abcam), and CD4 (ab237722: Abcam). Antigen visualization was performed using Astra dye (TheraNovis) for 10 minutes. The slides were then treated with Bond Epitope Retrieval 1 (AR9961: Leica Biosystems) for 20 minutes to remove bound antibodies before proceeding to the next step. The process from blocking step to antigen retrieval step was repeated for all antibody staining. After the final antigen retrieval step, the slides were counterstained by staining nuclei with DAPI (62248; ThermoFisher Scientific). The slides were coverslipped using ProLong Gold antifade reagent (P36930; Invitrogen).

Multiplexed stained slides were scanned at 20× magnification using Phenolmager™ HT (Akoya Biosciences). Representative images for training were selected in Phenochart™ Whole Slide Viewer (version 1.0.12, Akoya Biosciences), and algorithms were generated in inForm® Tissue Analysis software (version 2.6, Akoya Biosciences). Multispectral images were separated using the spectral library of inForm software. Each single cell was segmented based on DAPI staining. Phenotyping was then performed according to the expression site and intensity of each marker. After designating regions of interest (ROI) for analysis in tissue slides, the generated algorithms were applied identically for batch processing. Exported data was integrated and analyzed using phenoptr (Akoya Biosciences) and phenoptrReport (Akoya Biosciences) packages with R Studio (version 4.1.1). To measure the distance between two cells, the nearest cell was used for analysis. Subsequently, the proportion of other cell types within a 30 μm radius was calculated.

### 26. Statistical Analysis

All data was expressed as mean±standard deviation (SD) or standard error of the mean (SEM) for control and experimental samples. Multiple group comparisons were performed using analysis of variance (ANOVA), followed by Tukey's post-hoc test. Statistical significance was established using confidence intervals of 95% (*p<0.05), 99% (**p<0.01), 99.9% (***p<0.001), and 99.99% (****p<0.0001). Statistical analysis was performed using GraphPad Prism 9.5.0 (GraphPad Software, San Diego, CA, USA).

### [Experimental Results]

### 1. Design, Biosynthesis, and Physicochemical Characterization

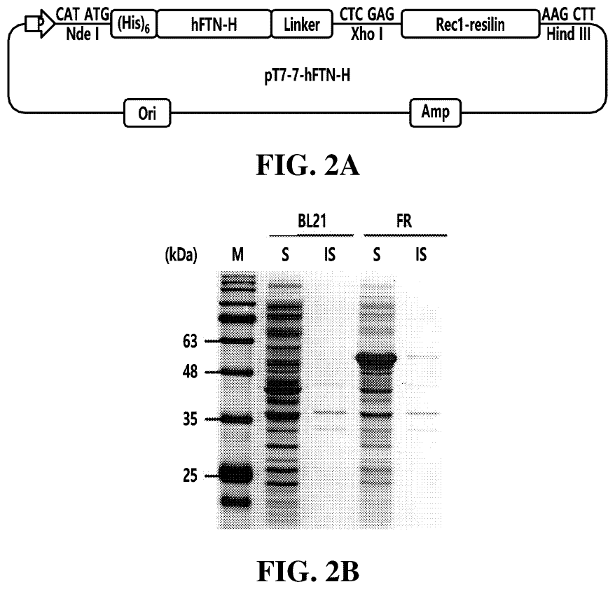

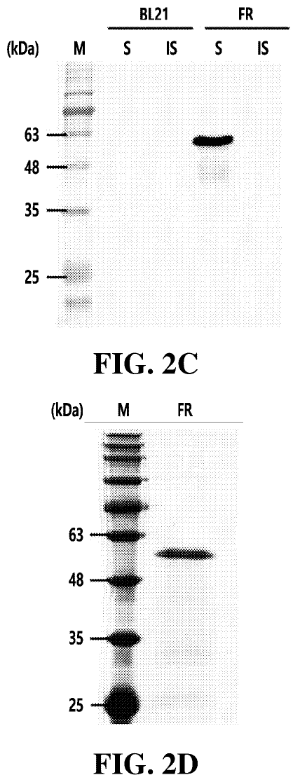

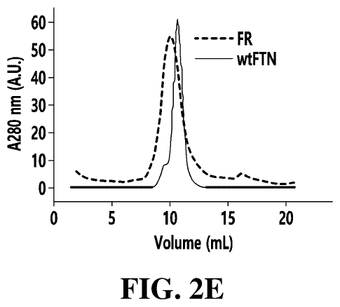

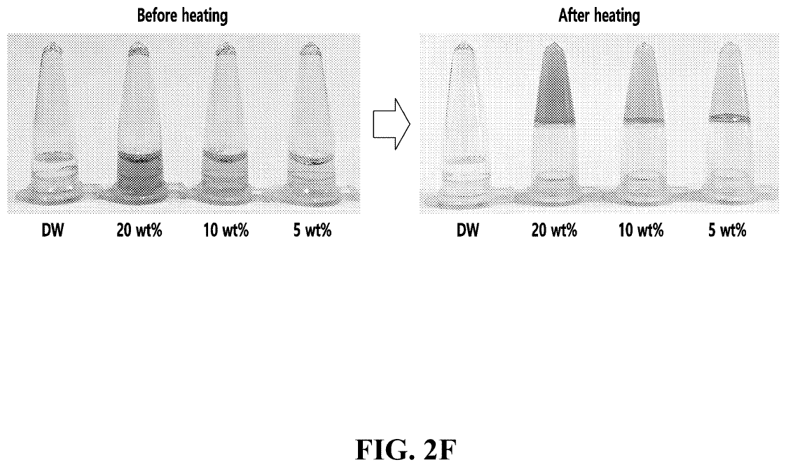

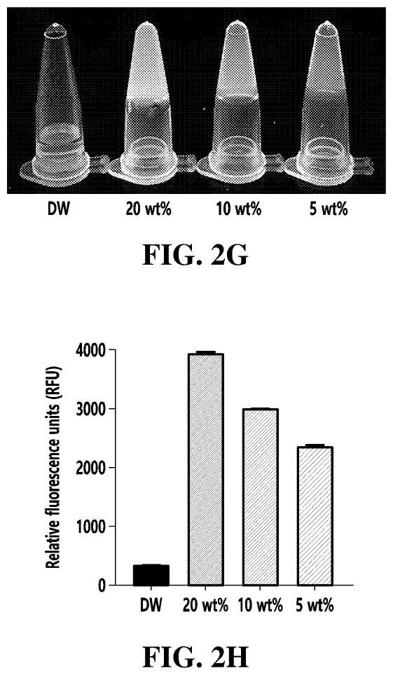

To synthesize hierarchical protein nanocrystal hydrogel, building block proteins composed of structural and non-structural domains were designed. Resilin, an elastic protein found in the wings, hinges, and tendons of insects and arthropods, was selected as the main component of the non-structural domain. Resilin provides excellent multi-stimulus responsiveness, rubber-like elasticity, and more than 92% resilience, conferring high structural flexibility and energy storage capacity required for jumping, flying, and active organ movements to specialized anatomical structures. In particular, the physical properties of rec 1-resilin, a recombinant resilin-like intrinsically disordered protein (IDP), are very similar to those of natural resilin encoded by exon I of resilin found in Drosophila melanogaster. Protein nanocages were selected to mimic hierarchically structured natural protein domains because protein nanocages self-assemble into highly organized supramolecular structures with uniform size distribution and provide sufficient mechanical stability. Among various protein nanocages, hFTN-H maintains excellent stability and crystal structure characteristics, even under a wide range of pH and temperature conditions. The building block protein of hierarchical hydrogel was genetically fused with the monomeric form of intrinsically disordered rec1-resilin and ferritin nanocage (FIGS. 1A and 2A).

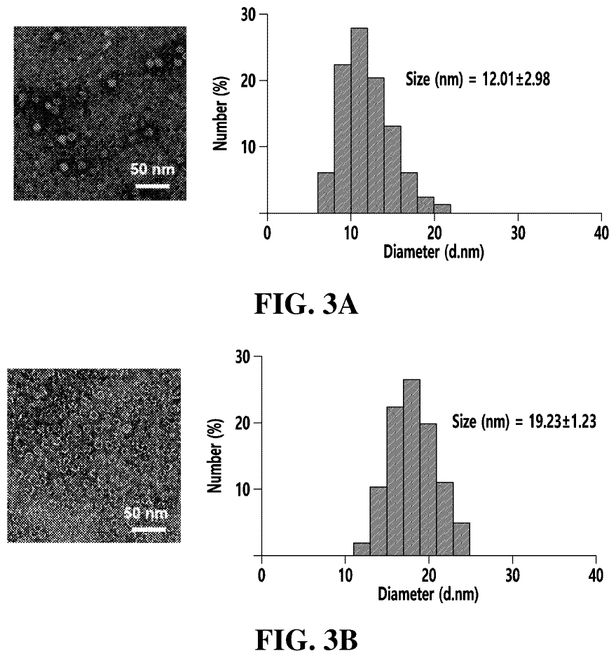

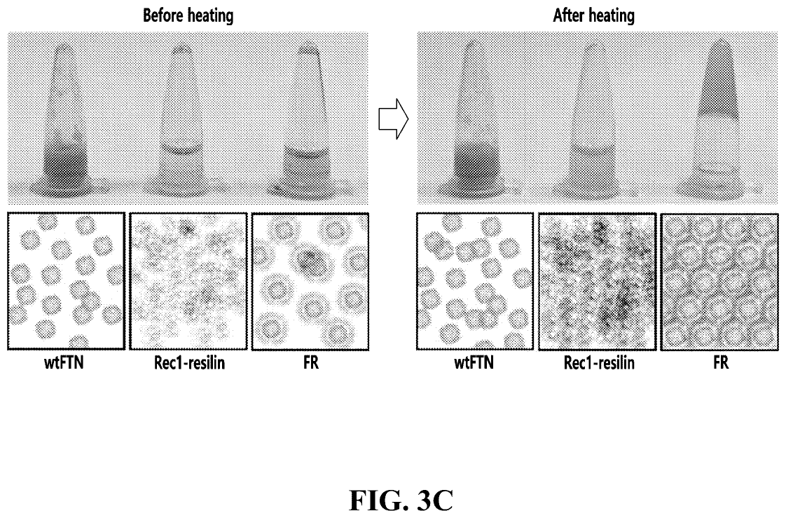

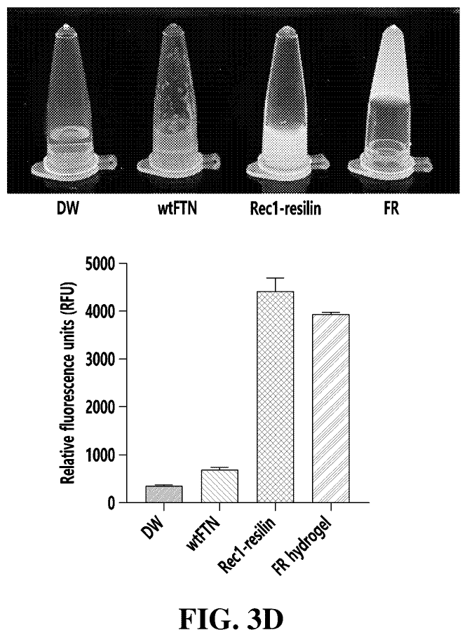

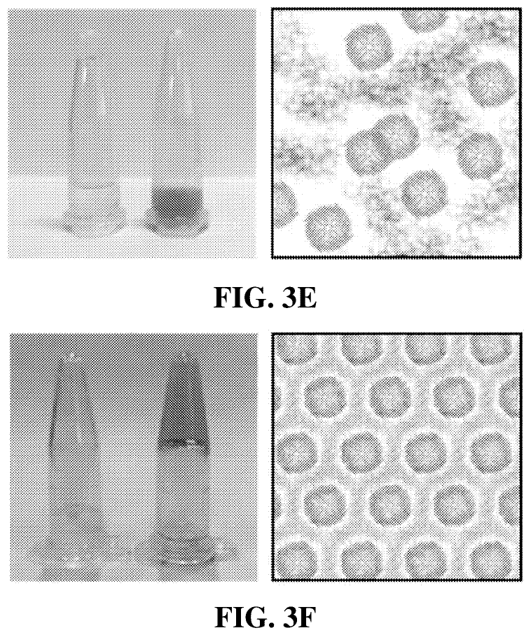

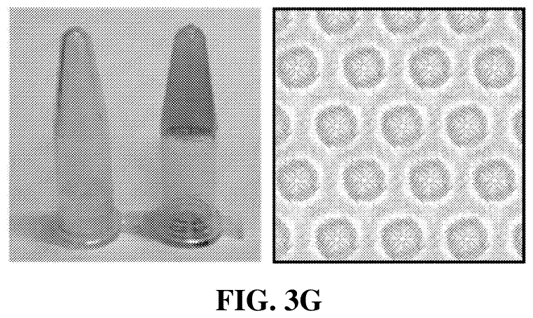

The recombinant building block protein, designated as FR, consists of ferritin nanocage crystals displaying high-density disordered rec1-resilin on the surface of the ferritin nanocage. This protein was successfully expressed in E. coli with 95.7% solubility and purified by Ni-affinity chromatography (FIGS. 2A to 2D). The oligomeric state and uniform nano-sized morphology of FR protein were observed using TEM, DLS, and SEC. Similar to the spherical morphology of wtFTN shown in FIG. 3A, FR also exhibited spherical particles with an average diameter of 19.39+1.23 nm (FIG. 3B). In vitro studies showed no new multimers or oligomers appeared compared to wtFTN through SEC elution profiles, and FR maintained nano-sized oligomeric state for more than 14 days, indicating structural stability (FIG. 2E). Collectively, the bioengineered FR building block protein was successfully produced in E. coli and indicates the presence of rec1-resilin on the surface of self-assembling structural ferritin nanocage.

### 2. Fabrication And Characterization of Hierarchically Structured Protein Nanocrystal Hydrogel

Generally, resilin-based hydrogels are formed through biochemical crosslinking networks of di- or tri-tyrosine bonds formed through various strategies. These strategies include (i) peroxidase, (ii) Mannich reaction, (iii) photochemical crosslinking mediated by photosensitizers [i.e., Ru(bpy)₃²⁺], and (iv) photo-Fenton reaction. This study demonstrated for the first time the formation of hydrogel from crosslinking of rec1-resilin through simple heat treatment. This methodological advancement not only contributes to the biocompatibility of the material but also significantly expands the possibility for in vivo applications.

According to previous studies, resilin protein exhibits multi-stimulus responses to temperature, pH, ionic, and light changes under aqueous conditions. In particular, full-length resilin forms into condensed spheres through thermal loss of bound water molecules. This process promotes additional hydrophobic interactions between tyrosine phenol aromatic groups of adjacent protein chains, resulting in di- or tri-tyrosine crosslinking. Since the thermal transition of resilin was measured to occur at approximately 50° C., the gelation temperature of FR was set at 55° C. Notably, as shown in FIG. 3C, 20 wt % FR solution was successfully converted to nanocrystal solid material after heat treatment. In contrast, solutions containing wtFTN or rec1-resilin did not undergo gelation. Furthermore, FR hydrogel emits blue fluorescence under UV irradiation, confirming the presence of di- or tri-tyrosine crosslinking (FIG. 3D). In contrast, control samples (i.e., deionized water or wtFTN) did not exhibit such fluorescence. Interestingly, rec1-resilin solution exhibited stronger fluorescence than FR hydrogel, suggesting more di- or tri-tyrosine crosslinking. This means that the same amount of rec1-resilin can form more di- or tri-tyrosine bonds than FR, but this is not sufficient to cause gelation. Consequently, the formation of FR protein hydrogel was achieved by imparting structural stability through coupling ferritin nanocage to rec1-resilin. Furthermore, individually expressed and mixed wtFTN and rec1-resilin proteins that were not genetically fused failed to induce such nanocrystal gelation after heat treatment (FIG. 3E).

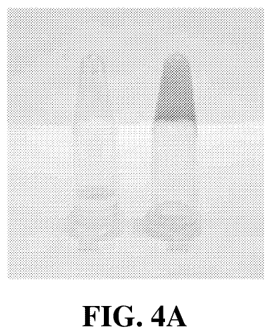

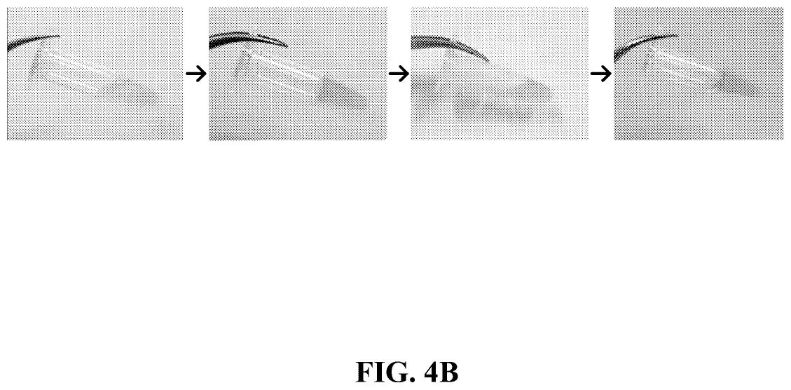

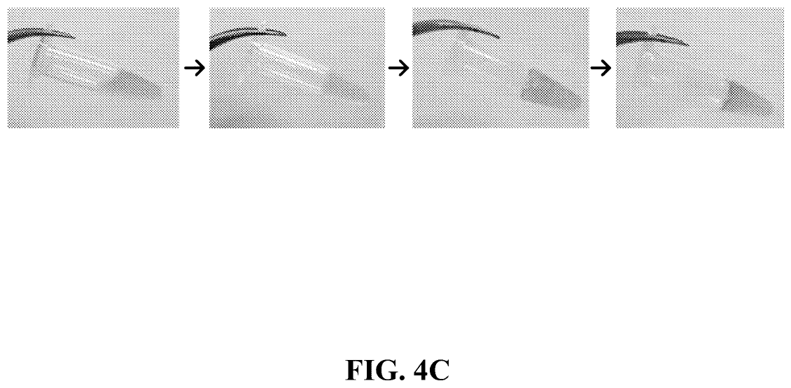

Human ferritin plays an important role in the physiological process of iron storage and undergoes self-oxidation through peroxidase reactions. Furthermore, Welch et al. revealed that the peroxidase activity of ferritin is caused by cysteine at position 90, which results in increased intermolecular di-tyrosine formation. Based on these previous studies, it was explored whether not only the structural influence of ferritin but also peroxidase activity could affect gelation. As shown in FIG. 3F, C90R FR solution successfully formed hydrogel, confirming that cysteine residues are not critical factors for hydrogel formation. Additionally, peroxidase non-reactive FR (C90R/C102A/C130A) with additional mutations in the peroxidase reaction sites of human ferritin also formed solid gel (FIG. 3G). Furthermore, Elvin et al. reported that 20 wt % rec1-resilin protein solution was optimal for forming solid gel through photochemical crosslinking, and hydrogel was not formed at concentrations below 10 wt %. However, FR hydrogel was successfully converted to hydrogel at concentrations of 20 wt %, 10 wt %, and even 5 wt % (FIGS. 2F to 2H). Additionally, thermally induced FR hydrogel could be formed even at 3° C., indicating potential as an in vivo injectable hydrogel (FIGS. 4A to 4C). Collectively, FR hydrogel was successfully produced through heat treatment even at very low concentrations. Coupling ferritin nanocage to elastic rec1-resilin is essential for the mechanical stability of the scaffold and provides important structural stability.

### 3. Rheological Properties, Micro-, Nano-, Molecular Structure, Swelling Kinetics, and Self-Healing Properties of Hierarchical Protein Hydrogel

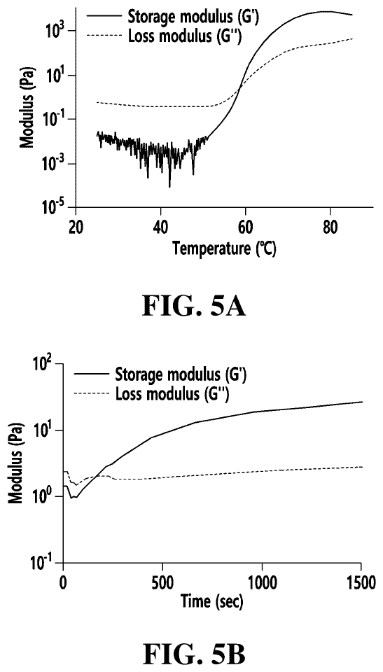

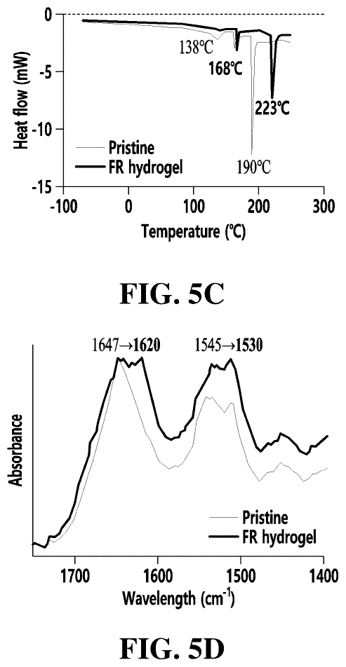

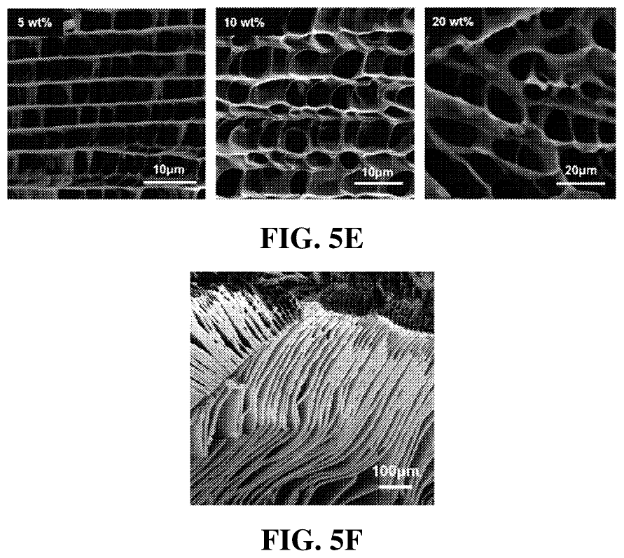

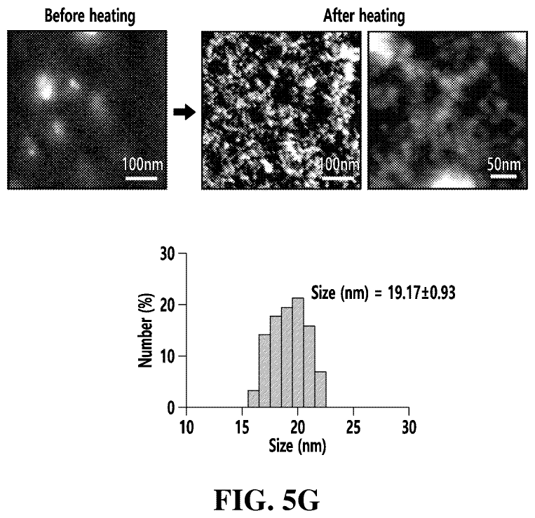

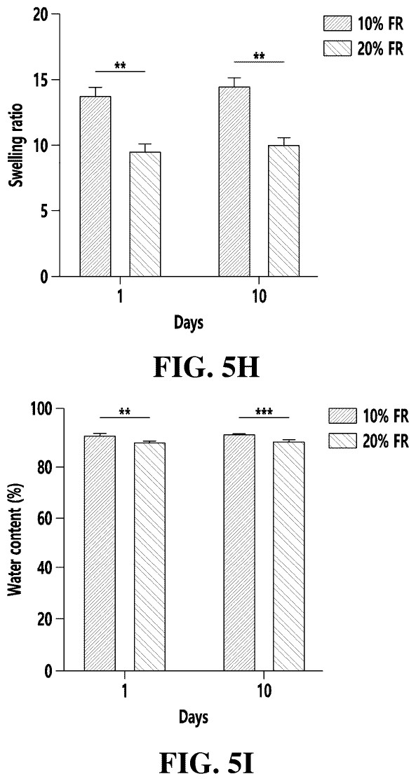

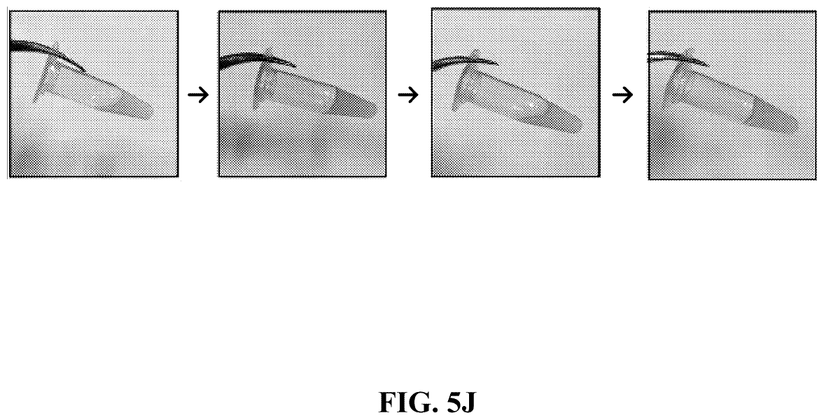

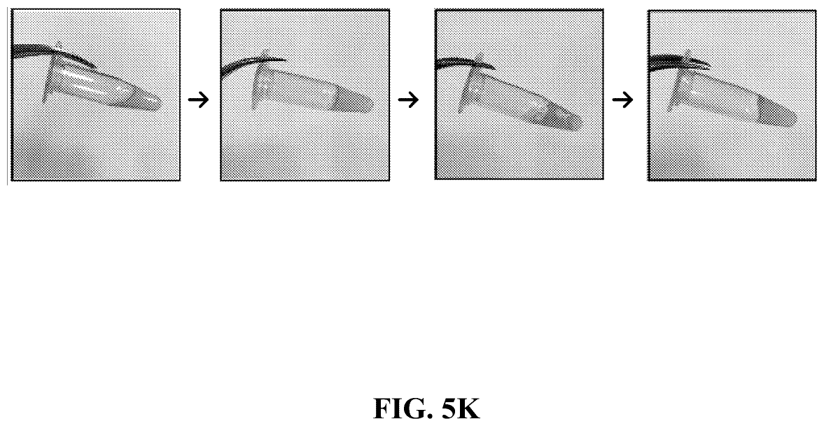

The rheological properties of hydrogels are very important because they affect the stability of hydrogel reservoirs in carrying materials such as cells or in in vivo environments. Therefore, gel transition during heat treatment was monitored and the viscoelastic mechanical properties of FR hydrogel were evaluated through dynamic oscillatory shear rheology using parallel plate geometry. Temperature sweep experiments showed temperature-dependent hydrogelation where G′ (storage modulus) exceeded G″ (loss modulus) between 50° C. and 60° C. FIG. 5A). Time sweep tests showed that FR hydrogelation occurs in a temperature- and time-dependent manner, with rapid gelation occurring within 3 minutes (FIG. 5B). Furthermore, the stiffness of FR hydrogel was considered sufficiently tuned to extracellular matrix (ECM) proteins (1 Pa˜1 kPa) and mammalian organs (100 Pa˜10 kPa).

To understand structural changes during FR protein gelation, FR solution and nanocrystal hydrogel were analyzed by DSC and FT-IR. As shown in FIG. 5C, DSC results of FR solution show two typical physical transition temperatures (138° C. and 190° C.). Considering that structurally more stabilized proteins (with highly organized structures) exhibit higher transition temperatures, FR hydrogel exhibits higher transition temperatures (168° C. and 223° C.). Additionally, in FT-IR spectra, relative shifts were observed from 1647 cm⁻¹ to 1620 cm⁻¹ in the amide I region and from 1545 cm⁻¹ to 1515 cm⁻¹ in the amide II region. These shifts indicate structural changes from random coil to β-sheet (1647 cm⁻¹ to 1620 cm⁻¹) and different vibrational modes of tyrosine side chains (1545 cm⁻¹ to 1515 cm⁻¹), indicating that FR protein forms more compact and ordered structures during the gelation process (FIG. 5D).

Next, the micro- and nanostructures of FR hydrogel were investigated. SEM images of the hydrogel shown in FIGS. 5E and 5F show three-dimensional scaffolds with uniform microporous morphology. Particularly, when separated, the hydrogel split into uniform internal porous structures along linear arrangements rather than random fragments. This suggests that the molecular-level structure of the hydrogel is based on intermolecular crosslinking of FR nanocrystals with hierarchical structure (FIG. 5E).

To further understand the structure of the hydrogel at the molecular level, AFM analysis was performed. Molecular topographic images of FR solution before and after heat treatment confirmed intermolecular crosslinking of FR nanocrystal cages during the gelation process (FIG. 5G). Before heat treatment, no typical surface morphology patterns were observed, but after heat treatment, FR solution formed elaborate nanoparticle patterns with a size of 19.17±0.93 nm, constituting a network. This suggests that thermally induced FR hydrogel consists of intermolecular crosslinking of each intact building block nanocage. Remarkably, the molecular structure of FR is comparable to natural full-length resilin. Natural full-length resilin consists of three domains: highly disordered regions of ‘exon I’ that confer elastic properties, ‘exon II’ containing chitin-binding domains, and structured ‘exon III’. These three domains form tightly packed spherical micelle particles of 20-30 nm size, constituting nanoscaffolds. This structure plays an important role in functions such as elasticity during energy storage and release. The nanostructure of FR particles observed in this study suggests that ferritin nanocage is similar to micelle structure and can function as a substitute for the exon III domain. Therefore, FR building block protein can be used as a biomimetic material of natural full-length resilin with energy conversion mechanisms.

Hydrogels with high water content have potential as ideal drug delivery vehicles due to their high water affinity and permeability, which enhance excellent biocompatibility and the ability to load and deliver hydrophilic cargo. Therefore, swelling kinetics were evaluated by monitoring water absorption of freeze-dried FR hydrogel immersed in PBS for 10 days. As shown in FIG. 5H, 10 and 20 wt % FR hydrogels swelled rapidly until reaching near equilibrium within 24 hours. Unfortunately, hydrogel composed of 5 wt % FR solution degraded after 10 days of swelling due to relatively low mechanical stability. Due to higher di- or tri-tyrosine crosslinking density, the swelling ratio of 20 wt % FR hydrogel at equilibrium was lower than that of 10 wt % FR hydrogel. While exhibiting excellent swelling grades, the water content of FR hydrogel was further evaluated (FIG. 5I). Generally, the water content of hydrogels is classified as low swelling grade (20-50%), intermediate swelling grade (50-90%), high swelling grade (90-99.5%), and superabsorbent hydrogel (>99.5%). Compared to other elastic proteins (e.g., elastin with only 5% polar amino acid residues), FR hydrogel was composed of rec1-resilin with 46% polar amino acid residues and protein nanocages that maintain highly porous structure and can contain up to 90% solvent. Therefore, water absorption of FR hydrogel reached macroscopic swelling (up to 93.56%), which is expected to facilitate high permeability and mass transfer required for various cellular functions.

Hydrogels with relatively soft yet self-healing properties ideally adapt to the movements of proliferating and migrating natural cells, enabling cell penetration in various directions while overcoming external stimuli or enzymatic degradation. Furthermore, in the development of injectable hydrogels, self-healing properties enable efficient energy dissipation under shear stress during injection and rapid self-recovery after injection. To investigate the reformation and self-healing properties of FR hydrogel, 10 wt % and 20 wt % FR hydrogels were crushed into very small pieces and then subjected to heat treatment. As a result, reformation of the hydrogel was successfully achieved, which may be attributed to hydrogen bonding and x-x interactions of tyrosine residues within the nanocrystal hydrogel (FIGS. 5J and 5K). According to previous studies, hydrogen bonding and I-x interactions of tyrosine residues in resilin play important roles in reassembling proteins into cross-β-like fibrous networks. These interactions result in thermodynamically controlled self-healing behavior. Additionally, recent studies have reported that hierarchical supramolecular organization of separated soft/hard domains plays important roles in providing rigid structure and maintaining self-healing capability. Therefore, the reversible nature of formation and destruction of non-covalent bonding interactions such as hydrogen bonding and I-x interactions within hierarchical supramolecular organization can enable thermodynamic self-healing properties of FR hydrogel. Moreover, FR hydrogel exhibits self-healing properties under physiological temperature conditions (37° C.), emphasizing its suitability for in vivo environments (FIG. 4).

### 4. Isolation and Characterization of sEV

The spleen is a representative SLO, the largest lymphoid organ in the human body, and contains one-quarter of the body's lymphocytes. The spleen performs two functions according to two distinct compartments: red pulp and white pulp. The red pulp consists of blood-filled venous sinuses and serves as a mechanical filtration system that removes foreign substances and damaged cells from the blood. The white pulp contains lymphoid tissue mainly composed of lymphocytes and plays a role in initiating immune responses against invading pathogens. It was investigated whether sEVs can reflect the organ itself and essentially contain characteristics similar to lymphoid organs. Based on this possibility, the potential of sEVs to induce ELS-like structures in immune cells was explored.

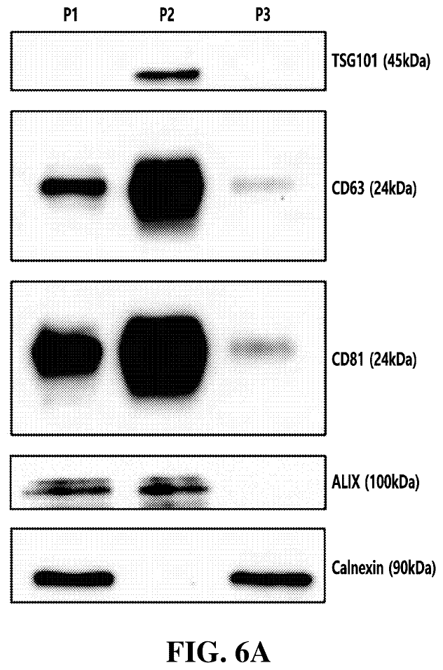

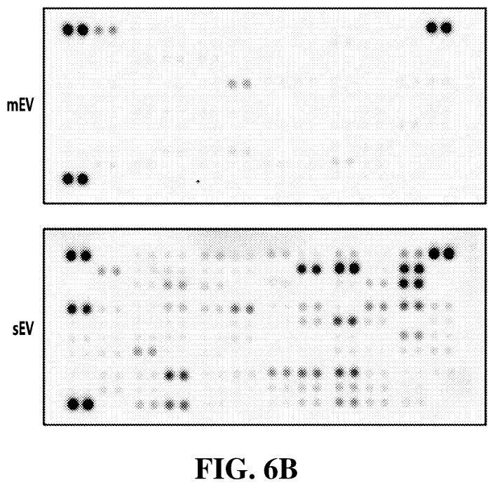

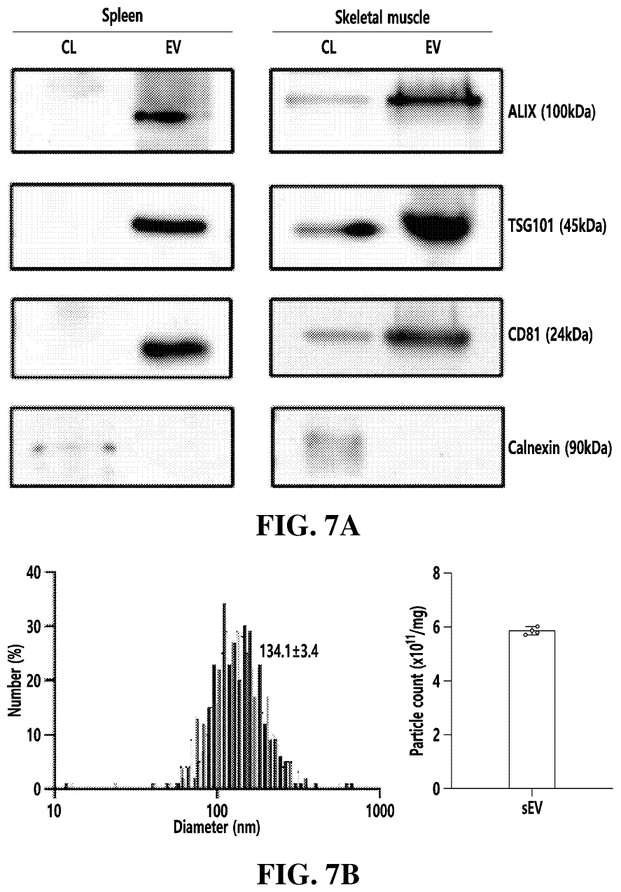

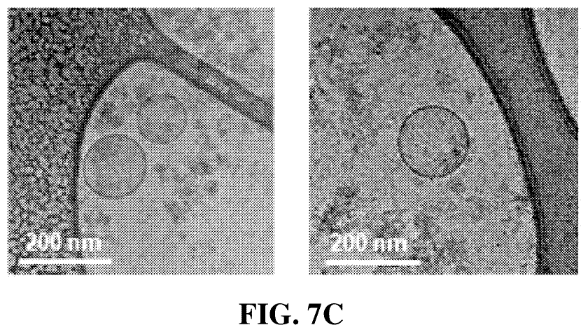

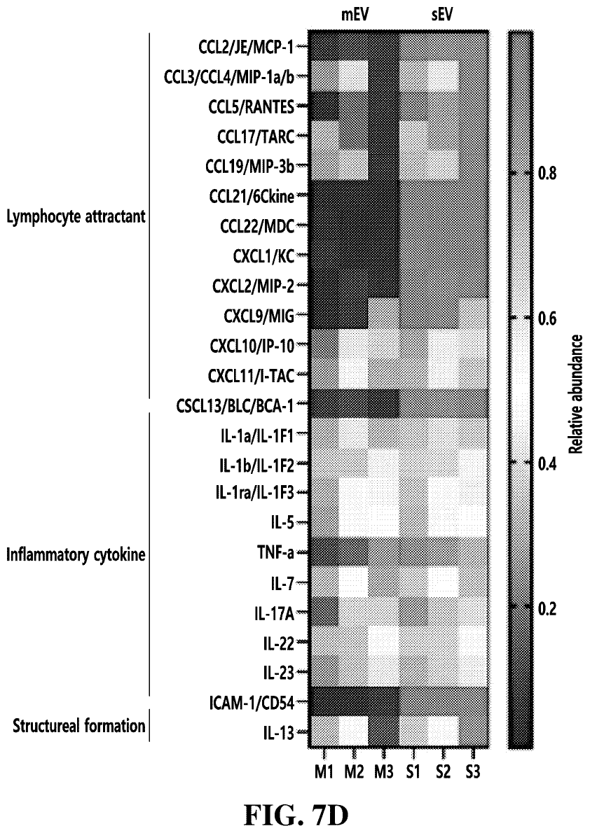

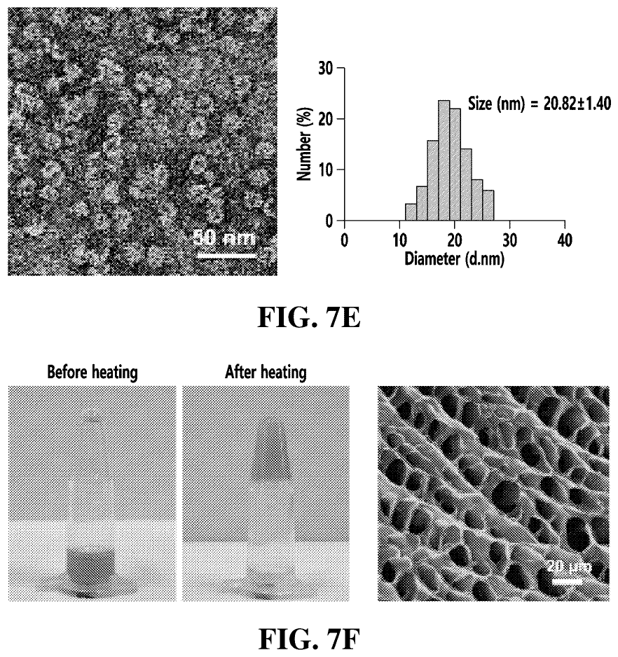

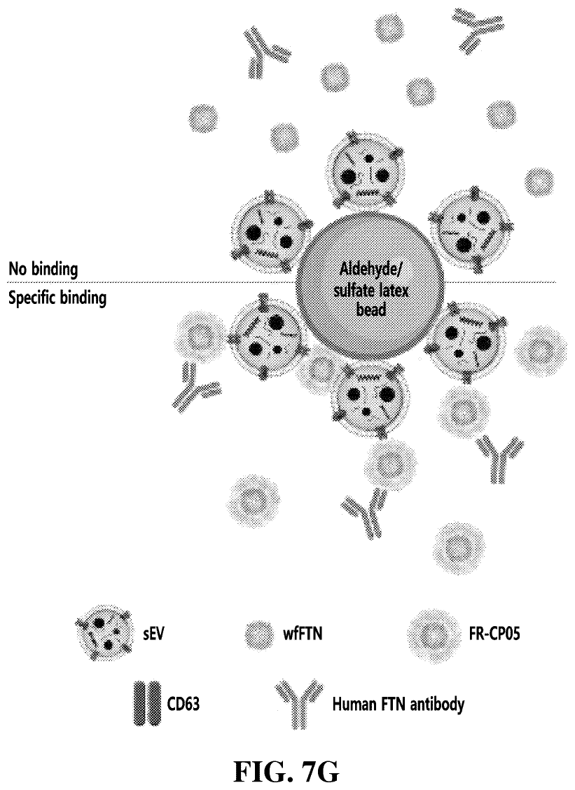

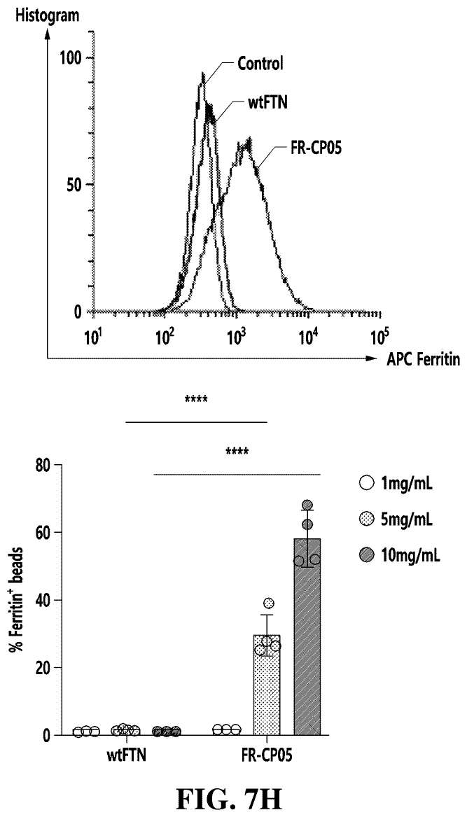

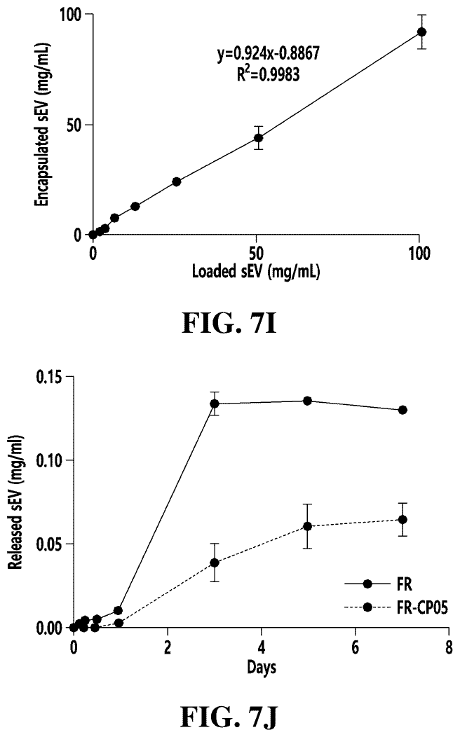

Since there is no established protocol for isolating organ-derived EVs, attempts were made to obtain sEVs using three different protocols based on previous organ- or tissue-derived EV studies. The most desirable protocol was selected through immunoblotting for several EV markers (e.g., tumor susceptibility gene 101 (TSG101), CD63, CD81, and Alix) and negative marker calnexin, and this method was used in the remaining studies (FIG. 6A). EVs were also isolated from mEV and used as a comparative control. As shown in FIG. 7A, SEVs and mEVs showed distinct EV marker profiles, whereas lysates obtained from their derived cells responded only to calnexin. Additionally, nanoparticle tracking analysis (NTA) and Cryo-TEM analysis showed that sEVs exhibited uniform circular morphology with a diameter of approximately 134.1 nm and a concentration of approximately 5.78×10¹¹ particles/mg (FIGS. 7B and 7C).

Proteome Profiler Mouse XL Cytokine array was used to further characterize sEVs and their protein profiles. A total of 111 proteins were detected in the array, of which 26 were directly linked to ELS induction (FIG. 6B). Most of these 26 cytokines were significantly increased in sEVs compared to mEVs and were grouped into three categories according to how these cytokines contribute to ELS formation (FIG. 7D). Most cytokines abundant in sEVs are lymphocyte attractants such as CXCL13 and CCL21, which are important for the generation and induction of ELS. Other abundant proteins include soluble intercellular adhesion molecule-1 (sICAM-1) and interleukin-13 (IL-13), which play important roles in structural aspects of ELS formation. Additionally, sEVs showed a tendency to increase levels of inflammatory cytokines, although this difference was not statistically significant except for

TNF-alpha and IL-23. These results suggest that sEVs deliver various protein cargo that directly indicates ELS induction potential while exhibiting characteristics of EVs.

### 5. Fabrication and Characterization of Hierarchical Protein Hydrogel Capable of Loading sEV

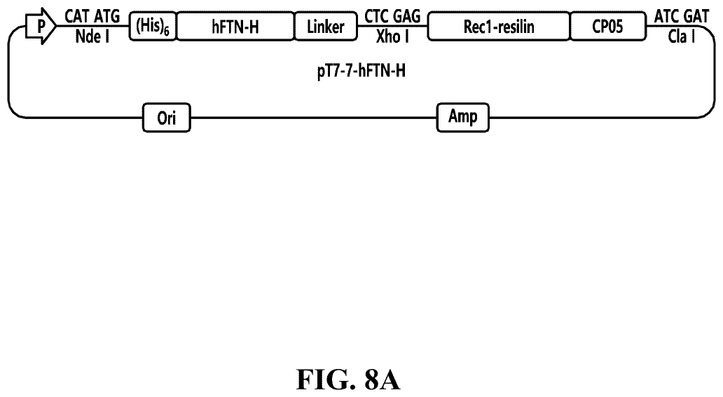

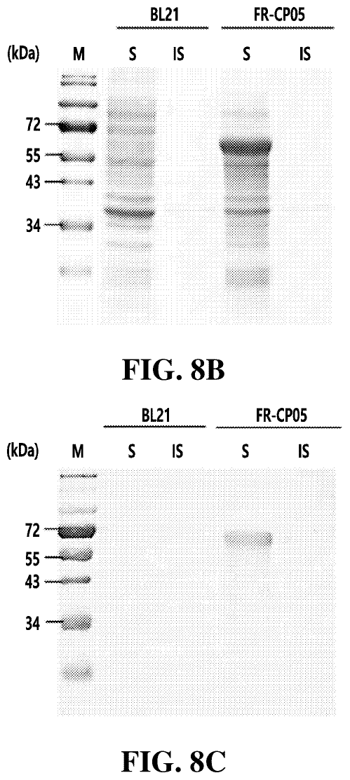

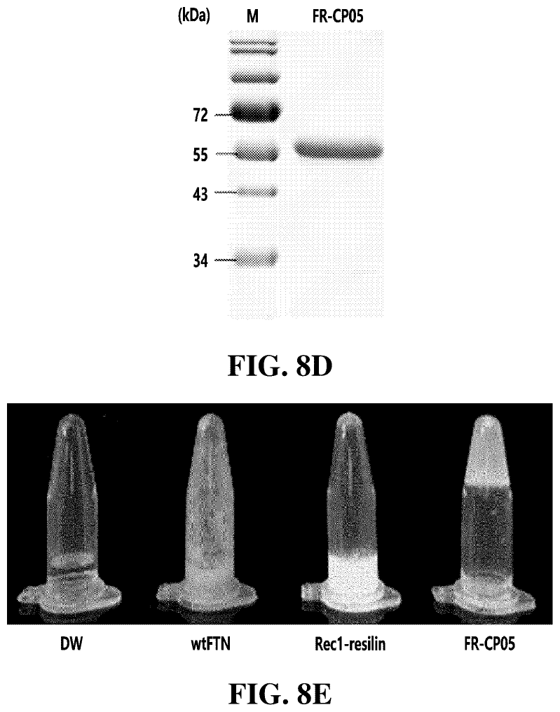

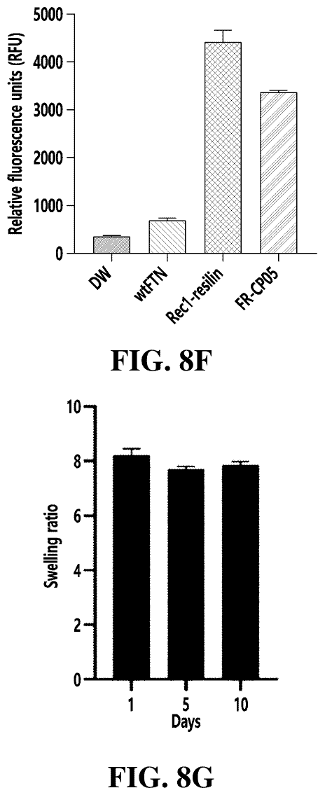

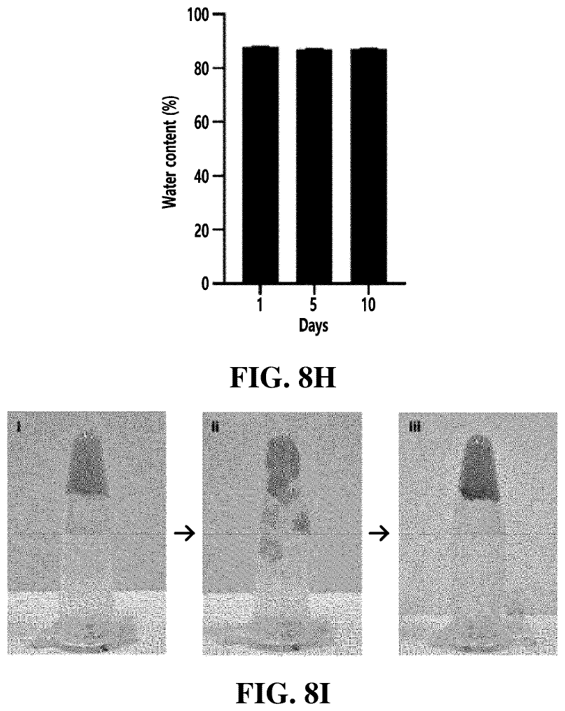

To fabricate a hydrogel that loads EVs, a CP05 peptide sequence (CRHSQMTVTSRL, SEQ ID NO: 2) capable of specifically capturing EVs by binding to CD63 was inserted into the C-terminus exposed on the surface of FR nanocage (FIG. 8A). The bioengineered FR-CP05 nanocage was successfully expressed in E. coli and formed spherical nanocages with a size of 20.82+1.40 nm (FIGS. 8A to 8D and FIG. 7E). FR-CP05 nanocage exhibited hierarchical microporous structure, swelling properties with high water content, and self-healing capability under heat treatment, showing characteristics similar to the original FR hydrogel (FIGS. 8E to 81, and FIG. 7F).

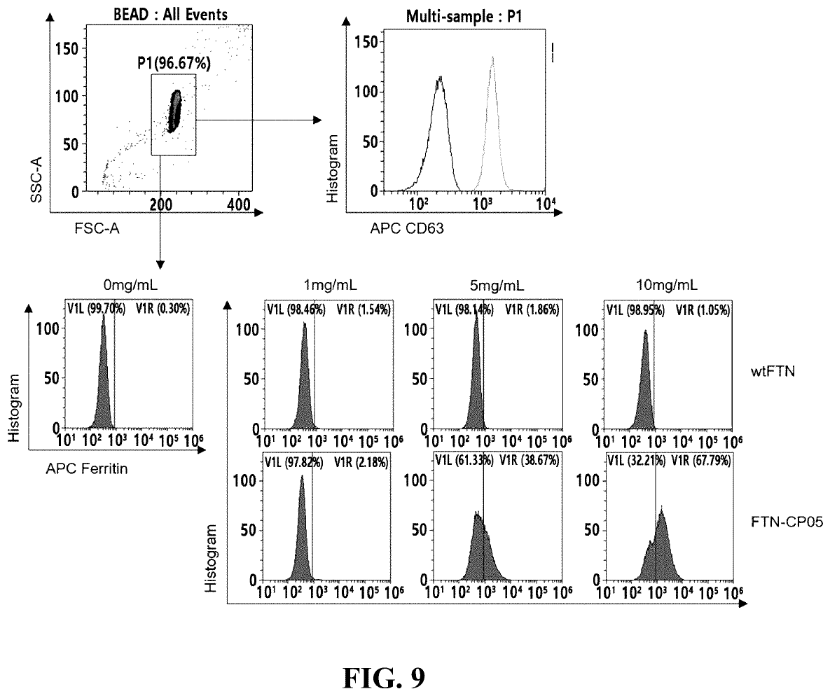

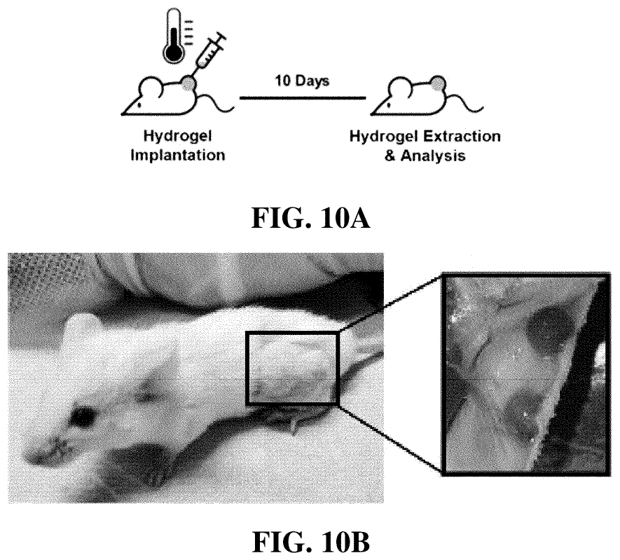

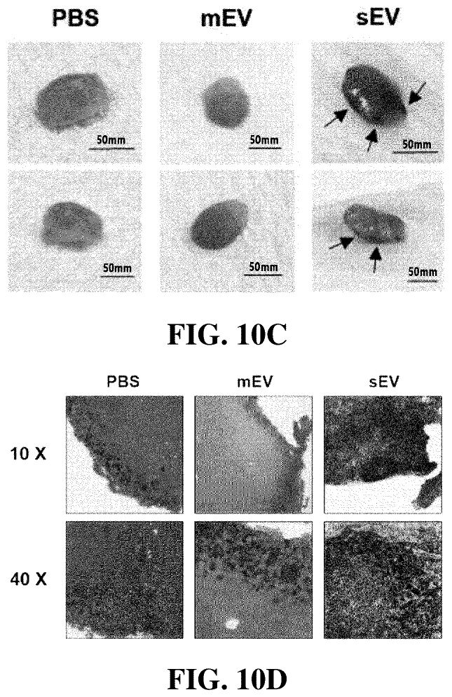

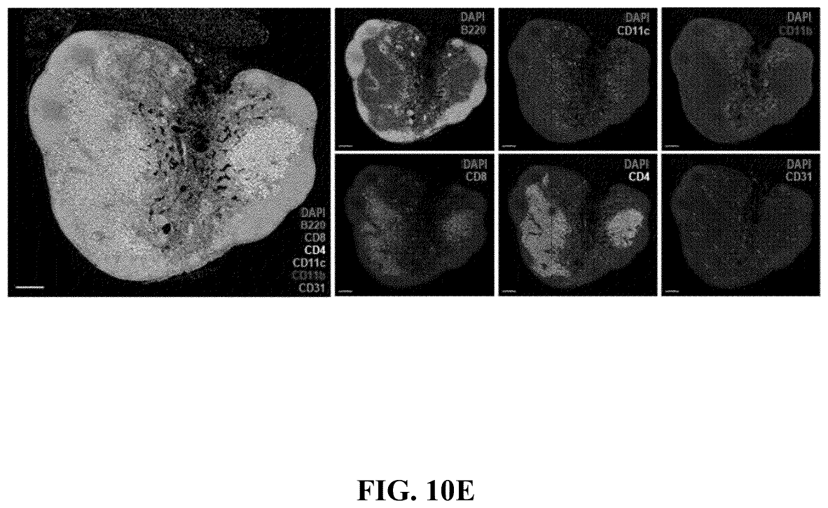

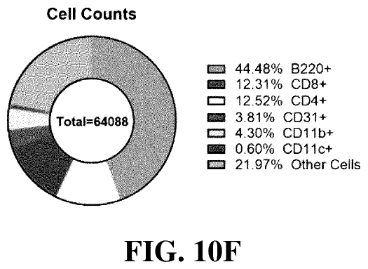

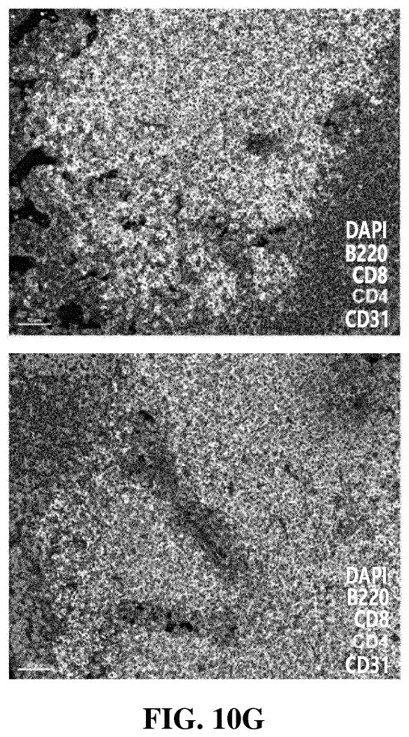

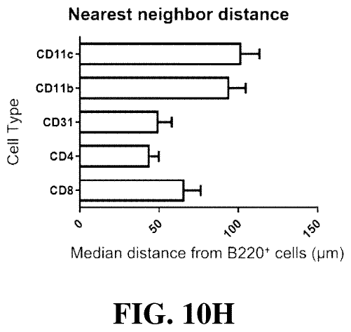

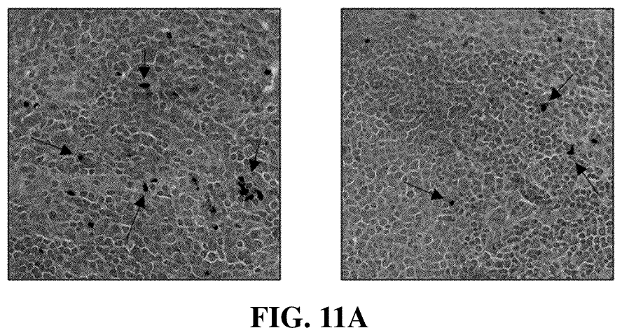

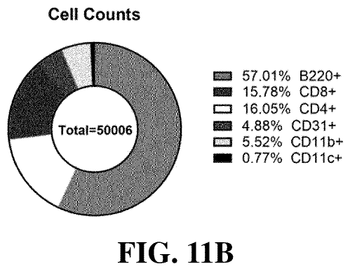

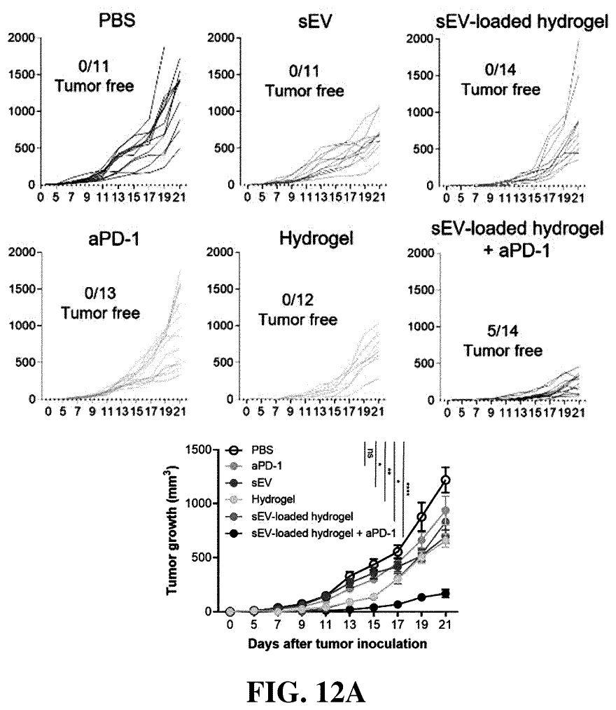

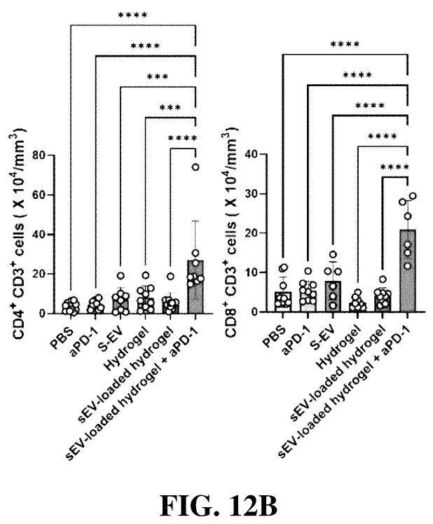

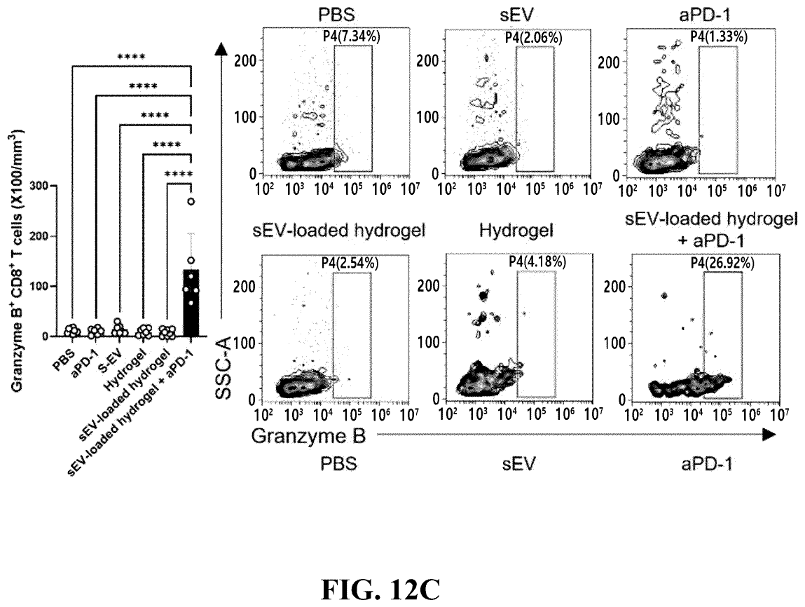

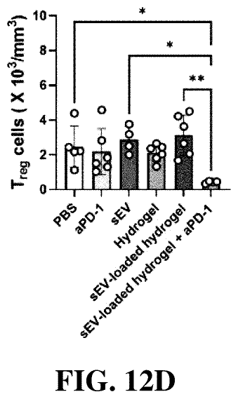

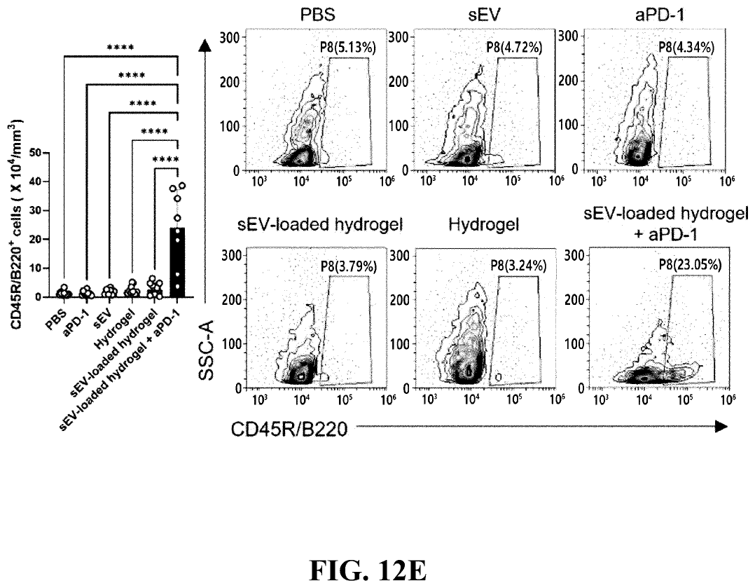

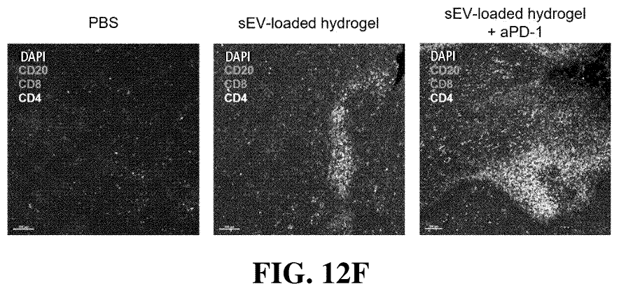

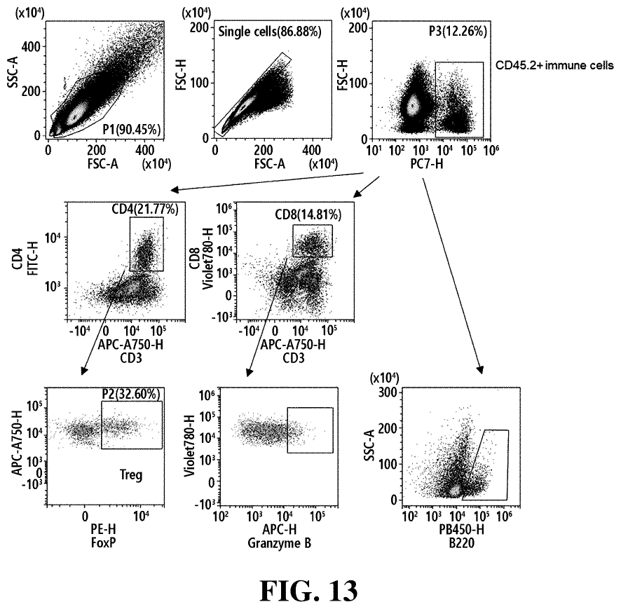

To verify the ability of FR-CP05 to capture sEV, binding experiments were performed using aldehyde/sulfate latex beads coated with sEV (FIG. 7G). As shown in FIGS. 7H and 9, FR-CP05 nanocage bound to sEV in a concentration-dependent manner, whereas wtFTN did not show such binding affinity. Additionally, the loading capacity of FR-CP05 hydrogel was analyzed using Cy5-labeled sEV, confirming that the loading profile increased linearly as FR-CP05 nanocage continuously bound to sEV (FIG. 71).

Furthermore, the time-dependent release performance of sEV loaded in FR-CP05 hydrogel was investigated (FIG. 7J). FR-CP05 hydrogel maintained sEV release remarkably sustained and stable, whereas the group without binding chemistry showed rapid release on day 2. In summary, integrating EV-binding peptide into FR hydrogel did not affect thermally induced gel transition, microstructure, swelling, or self-healing capabilities. However, this enabled high-level sEV loading and long-term release within the hydrogel. This advancement can serve as a physical scaffold and effective delivery platform for sEV in ELS formation.

### 6. Evaluation of Artificial ELS Formation and Immune Cell Profiling in ELS-Inducing Hydrogel

Next, whether sEV-loaded hydrogel could induce ELS formation in an in vivo mouse model was explored. After subcutaneous injection of hydrogel into BALB/c mice, the hydrogel was removed and analyzed 10 days later (FIG. 10A). At this time point, the hydrogel existed visibly at the injection site (FIG. 10B), and sEV-loaded hydrogel showed signs of angiogenesis (FIG. 10C). Considering the role of cytokines such as CCL2 and CXCL13 in angiogenesis, the enhanced angiogenic capability may result from the abundance of sEV-derived structural proteins and cytokines (e.g., CCL2 and CXCL13) compared to mEV. Additionally, H&E staining images of each hydrogel showed significant differences in cell infiltration (FIG. 10D). Hydrogel without sEV showed limited cell infiltration, whereas sEV-loaded hydrogel showed almost complete cell infiltration and aggregation, as well as angiogenesis with the presence of red blood cells (FIG. 11A).

To confirm whether hydrogel containing sEV formed ELS structures, the hydrogel was analyzed by multiplex IHC for representative ELS markers (B220, CD11c, CD11b, CD8, CD4, and CD31) to detect lymphocyte aggregation. ELS showing typical germinal center-like structures were confirmed by the presence of dense networks where B220+ B cells were surrounded by T cells (CD4+ and CD8+ cells). Additionally, staining results for CD11c (dendritic cell marker), CD11b (macrophage marker), and CD31 (endothelial cell marker) confirmed high infiltration of dendritic cells and macrophages and formation of endothelial vessels that assist ELS formation within the hydrogel (FIG. 10E). Quantitative analysis of these markers highlighted the relative abundance of each cell type (FIGS. 10F and 11B), showing that B220+ B cells were the most dominant group, accounting for approximately 45% of the total cell population and 57% of the stained cell population. CD4+ and CD8+ T cells accounted for 12.52% and 12.32% of the total cell population, and 16.05% and 15.87% of the stained cell population, respectively.

To confirm the presence of germinal center-like structures within the hydrogel, aggregation patterns of B cells and T cells were confirmed in magnified images of multiplex IHC slides (FIG. 10G). Nearest neighbor distances were calculated based on the distance between each cell type and B220+ B cells (FIG. 10H). The closest cell group was CD4+ T cells, followed by CD31+ endothelial cells, and CD8+ T cells were the third closest. This suggests that immune cells infiltrated the hydrogel through CD31+ high endothelial venules (HEV). This observation is consistent with previous studies showing that clonal expansion of CD4+ T cells is associated with high density of B cells. Germinal center-like ELS composed of aggregation of B cells and T cells are positively associated with improved prognosis in various cancers. Collectively, these results demonstrate that sEV-loaded hydrogel can effectively induce ELS formation, indicating significant potential for altering TME to support immune cell recruitment and function.

7. Enhanced Antitumor Effects of sEV-Loaded Hydrogel-Based Artificial ELS Formation Combined with ICB Therapy

In current medicine, ICB therapy is considered an important pillar and game-changing treatment in immuno-oncology. However, most patients do not show positive responses to ICB treatment for various reasons, one of which is attributed to the immune evasion state of tumors. Dysfunctional immune profiles or lack of various T cells, macrophages, and B cells are known to affect the prognosis of ICB therapy. In addition, maintaining an ‘immune-activated’ phenotype in tumors is positively correlated with ICB response. Since ELS is directly related to immune cell recruitment and functional enhancement, inducing ELS is rapidly becoming an attractive tool in the field of cancer immunotherapy. This approach shows high correlation with improved ICB response. Therefore, whether sEV-loaded hydrogel-based ELS formation could enhance the efficacy of ICB was explored.

First, mice were inoculated with CT26 tumor cells, a PD-1 resistant tumor cell line, and then sEV-loaded hydrogel was injected around the tumor. After hydrogel implantation, αPD-1 antibody was administered every 2 days. Remarkably, mice that received sEV-loaded hydrogel implantation together with PD-1 antibody therapy showed dramatically reduced tumor progression compared to other groups, with more than one-third of these mice reaching a tumor-free state (FIG. 12A). To analyze immune cell populations within tumor tissue, tumors were extracted. Immune cell infiltration was then investigated through flow cytometric analysis (FIG. 13). In the ELS-inducing hydrogel and PD-1 antibody group, increased numbers of CD4+ and CD8+ T cells per tumor volume were observed (FIG. 12B). Among CD8+ T cell subpopulations, granzyme B+ populations were more abundant in the ELS-inducing hydrogel and PD-1 antibody group (FIG. 12C). In contrast, Treg cells were significantly decreased (FIG. 12D). Interestingly, abundant presence of CD45R/B220+ B cells was confirmed (FIG. 12E). This supports previous studies showing the presence of tumor-infiltrating B cells in TME within ELS and their important role in tumor immune regulation, especially when combined with ICB treatment.

Multiplex IHC staining results of tumor tissue showed that the ELS-inducing hydrogel implantation group receiving PD-1 antibody treatment had significantly increased infiltration of CD20+ B cells, which was consistent with flow cytometric analysis data. This group also showed significantly increased CD4+ and CD8+ T cells within the TME compared to other groups (FIG. 12F). Particularly, B cells formed numerous aggregates in close proximity to T cells, indicating extensive immune cell infiltration, activation, and reorganization within the TME. This aggregation tendency of immune cells indicates that ELS occurred within the TME, suggesting that ELS-inducing hydrogel was implanted around the tumor and had already been degraded at this time point. Overall, ELS-inducing hydrogel improved the immune environment around the TME and promoted an ‘immune-activated’ phenotype within tumors through combination with ICB therapy. Therefore, the research results demonstrate that ELS-inducing hydrogel combined with ICB therapy can provide sufficient immunity to overcome tumor burden.

While the embodiments have been described with reference to the limited drawings, it will be apparent to one of ordinary skill in the art that various alterations and modifications can be made from the above description. For example, suitable results may be achieved if the described techniques are performed in a different order, and/or if components in a described system, architecture, device, or circuit are combined in a different manner, or replaced or supplemented by other components or their equivalents.

Therefore, other implementations, other embodiments, and equivalents to the claims are also within the scope of the following claims.

1. A composition for immune cell activation, comprising secondary lymphoid organ (SLO)-derived extracellular vesicle as an active ingredient,
wherein the extracellular vesicle is encapsulated in a hydrogel.

2. (canceled)

3. The composition of claim 1, wherein the hydrogel is formed by crosslinking of ferritin-resilin (FR) proteins.

4. The composition of claim 3, wherein each FR protein is composed of human ferritin heavy chain protein having recombinant rec1-resilin linked to C-terminus of the human ferritin heavy chain protein via a linker.

5. The composition of claim 4, wherein the linker comprises an amino acid sequence in which an amino acid sequence of SEQ ID NO: 1 is repeated 1 to 5 times.

6. The composition of claim 3, wherein each FR protein has a CP05 peptide linked to C-terminus of the FR protein.

7. The composition of claim 6, wherein the CP05 peptide comprises an amino acid sequence of SEQ ID NO: 2.

8-14. (canceled)

15. A pharmaceutical composition for cancer immunotherapy, comprising SLO-derived extracellular vesicle as an active ingredient.

16. The pharmaceutical composition of claim 15, wherein the pharmaceutical composition is for use in combination administration with an immune checkpoint inhibitor.

17. The pharmaceutical composition of claim 15, wherein the pharmaceutical composition further comprises an immune checkpoint inhibitor.

18. The pharmaceutical composition of claim 15, wherein the extracellular vesicle is encapsulated in a hydrogel.

19. The pharmaceutical composition of claim 18, wherein the hydrogel is formed by crosslinking of FR proteins.

20. The pharmaceutical composition of claim 19, wherein each FR protein is composed of human ferritin heavy chain protein having recombinant rec1-resilin linked to C-terminus of the human ferritin heavy chain protein via a linker.

21. The pharmaceutical composition of claim 20, wherein each FR protein has a CP05 peptide linked to C-terminus of the FR protein.

22. A method of preparing an FR nanostructure comprising SLO-derived extracellular vesicle, the method comprising:
(1) preparing an FR protein having a structure of [N-terminus—human ferritin heavy chain—linker—Rec1-resilin—CP05 peptide—C-terminus];
(2) mixing the FR protein with distilled water or phosphate-buffered saline (PBS) to prepare an FR solution; and
(3) mixing extracellular vesicle isolated from SLO with a 10-20 weight percent (wt %) FR solution.

23. The method of claim 22, further comprising (4) performing heat treatment on a mixed solution of step (3) to provide an FR nanostructure in a hydrogel formulation.

24. The method of claim 23, wherein the heat treatment of step (4) comprises applying heat of 50° C. or higher for 3 minutes or longer.


SEQUENCE LISTING (2 sequences)

SEQ ID NO: 1 - protein, 5 aa, synthetic construct
GGGGS

SEQ ID NO: 2 - protein, 12 aa, synthetic construct
CRHSQMTVTSRL